(19) United States
(12) Patent Application Publication
(10) Pub. No.: US 2023/0123863 A1
(43) Pub. Date: Apr. 20, 2023

(54) A GLIA CELL AND NEURON CO-CULTURE SYSTEM AND METHOD

(71) Applicant: National Yang-Ming University, Taipei (TW)

(72) Inventors: Yi-Hsuan Lee, New Taipei City (TW); Chia-Chi Hung, Keelung (TW); Pei-Chien Hsu, New Taipei City (TW); Yu-Jie Huang, New Taipei City (TW)

(21) Appl. No.: 17/281,979

(22) PCT Filed: Jun. 30, 2020
(86) PCT No.: PCT/US2020/040378

§ 371 Date: Mar. 31, 2021

(51) Int. Cl. C12N 5/079

(52) U.S. Cl. CPC C12N 5/0622; C12N 2502/081; C12N 2320/10; C12N 2500/34; C12N 2500/32

(57) ABSTRACT

The present invention comprises a system and method for co-culturing glia cells and neurons in a combination of glia culture medium and neuron medium wherein the glia cells and neurons have cell morphology, cell reactions and/or cell interactions that exist in vivo after the co-culturing for up to about 21 days, up to about 30 days, up to about 40 days, up to about 45 days. Since the cell morphology, cell reaction and/or cell interaction of the co-culture are similar to those seen in vivo, the present system is capable of being configured as animal models for research, drug screening, testing and conducting clinical trials.

### CROSS-REFERENCE TO RELATED APPLICATIONS

The present patent application claims priority to PCT patent application No. PCT/US20/40378 entitled “A Glia Cell and Neuron Co-culture System and Method” filed on Jun. 30, 2020 and is hereby incorporated in its entirety.

### FIELD OF THE INVENTION

The present invention relates to the field of cell cultures. In particular, the present invention relates to a system and method for co-culturing glia cells and neurons that provides cell morphology, cell reactions and/or cell interactions of glia cells and neurons similar to those in vivo such that the present invention is capable of being configured as animal models for research, drug screening, testing and conducting clinical trials.

### BACKGROUND OF THE INVENTION

Co-culture media are commonly used in the study of biological interactions between cells for simulating in vivo conditions. In functional studies especially, those skilled in the art often strive to culture cells in vitro that mimic the morphology and physical as well as physiological behavior of cells in vivo. In the culturing of brain cells, it is important to note that in vivo, neurons develop and mature surrounded by glia cells, such as astrocytes, microglia, and oligodendrocytes. As neurons and glia cells mature, processes form between the cells, linking them in close physical proximity, and physiological interactions occur via chemicals released from the cells.

There exists prior art that culture purified neurons and/or glia cells in vitro that result in cell morphology substantially different from that of cells in vivo, thus rendering them inaccurate approximations of in vivo conditions, less fit for use in studies.

For example, Zhang et al. purified astrocytes and neurons and cultured each cell type separately in astrocyte culture medium and neuronal culture medium, respectively, before culturing the two types of cells in neuronal culture medium, comprising 97% Neurobasal™ medium supplemented with 2% B-27, 100 U/mL penicillin, and 100 μg/mL streptomycin. In U.S. Pat. No. 10,093,898 B2, Barres et al. discloses an astrocyte growth medium comprising 50% Neurobasal™, 50% DMEM, 100 units/mL penicillin, 100 μg/mL streptomycin, 1 mM sodium pyruvate, 2 mM L-glutamine, lx SATO, 5 μg/mL NAC, and 5 ng/mL HBEGF. Also disclosed is a neuron growth medium for rat retinal ganglion cells and human fetal neurons, comprising 16 mL DMEM, 4 mL dH₂O, 200 μL 0.5 mg/mL insulin, 200 μL 100 mM pyruvate, 200 μL 100× penicillin/streptomycin, 200 μL 200 mM L-glutamine, 200 μL 100× SATO, 200 μL 4 μg/mL thyroxine (T3), 400 μL NS21-Max, and 20 μL 5 mg/mL NAC. The disclosure of Barres et al. lacks B-27 supplement, a supplement beneficial for long term culture of neural cells. Though rat neurons and astrocytes were co-cultured in a co-culture medium in both the disclosures of Zhang et al. and Barres et al., the purified neurons and astrocytes were first cultured separately in a neuron medium and astrocyte medium, respectively, thus the neurons and astrocytes were unable to develop in each other's presence, and unable to achieve the morphology and physical and physiological attributes of in vivo cells.

In addition, El et al. discloses a method of co-culturing cortical neurons and astrocytes obtained from rats in first a primary cortical neuronal-astrocyte cell culture medium comprising Modified Essential Medium (MEM) (without L-glutamine, with essential amino acids), 5% heat-inactivated fetal calf serum, 5% heat-inactivated horse serum, 2 mM glutamine, 3 mg/mL glucose, 2% B-27 supplement, and 0.5% penicillin/streptomycin (100 U/mL penicillin, 100 μg/mL streptomycin), then transferring the cells to a growth medium comprising MEM-EAGLE (without L-glutamine, with essential amino acids), 5% heat-inactivated fetal calf serum, 2 mM glutamine, 5 mg/mL glucose, 2% B-27 supplement, 0.5% penicillin/streptomycin, and 0.8% GlutaMax™ (100×). Though neurons and astrocytes are co-cultured together, the growth medium used by El et al. does not contain Neurobasal™ to support neuronal maturation.

Although there are existing cell culture media that comprise a combination of DMEM and Neurobasal™, these media cultures have only been used for culturing mainly stem cells and promoting their differentiation to mature into desired cell types rather than for co-culturing glia cells and neurons harvested from brains of mammals. Therefore, these existing stem cell based cultures are used for an entirely different purposes and for different cell types as compared to the present invention. In addition, these existing stem cell culture media also contain compounds necessary for stem cell differentiation that would not be necessary and would even be detrimental to the system and method of the present invention since in vivo conditions do not contain these factors in the amounts disclosed in these prior art, and, therefore, the composition of these existing stem cell cultures are also substantially different from the present invention.

Therefore, there is a need for a glia cell and neuron co-culture system and method that provide cell morphology, cell reaction and cell interaction similar to those in vivo.

### SUMMARY OF THE INVENTION

A glia cell and neuron co-culture system comprising glia cells and neurons co-cultured in a combination of glia culture medium and neuron medium wherein the glia culture medium comprises Dulbecco's modified eagle medium (DMEM) and the neuron medium comprises Neurobasal™ medium. In an embodiment, the glia cells and neurons are obtained from mammal brains. In another embodiment, the glia cells and neurons have cell morphology, cell reaction and/or cell interaction that exist in vivo after the co-culturing for up to about 21 days, up to about 30 days, up to about 40 days, up to about 45 days. In an embodiment, the co-culture system comprises a 2D culture.

In an embodiment, the neuron medium further comprises about 1000 to about 2000 mg/L glucose and DMEM comprises about 1750 to about 2750 mg/L glucose with no glutamine. In another embodiment, the system of the present invention further comprising 50× B-27 Plus™ supplement and 100× GlutaMAX™ supplement. In an embodiment, the glia culture medium further comprising one or more antibiotic. In another embodiment, the glia culture medium further comprising one or more animal serum. In an embodiment, the system of the present invention further comprising 1×B-27 serum free supplement and 2 mM L-glutamine.

In an embodiment, the ratio of glia culture medium to the neuron medium is about 0.5:1 to about 4:1. In another embodiment, the ratio of glia culture medium to the neuron medium is about 0.5:1. In an embodiment, the ratio of glia culture medium to the neuron medium is about 1:1.2 to about 4:1.

In an embodiment, the glia cells and neurons are co-cultured together starting at days in vitro (DIV)-0 and the cells mature together for more than 2 days, 5 days, 10 days or 21 days. In another embodiment, the combination glia culture medium and neuron medium does not comprise factors that promote differentiation, reversion or transformation from one cell type to another cell type including retinoid acid, leukemia inhibitory factor or N2 supplement.

In an embodiment, the glia cells comprise the combination of any glia cells that exists in mammal brains. In another embodiment, the glia cells comprise the combination of glia cells that usually exists in mammal brains and at about the same percentage ranges as that exist in mammal brains. In an embodiment, the glia cell comprises astrocytes, microglia, oligodendrocytes, stem cells or a combination thereof wherein the stems cells comprise no more than about 95%, 90%, 80%, 70%, 60%, 50%, 40%, 30%, 20%, 10% or 5% of the cells.

The present invention also comprises a method for preparing the system of the present invention, comprising the steps of (i) triturating the cerebral cortex obtained from an animal in the glia culture medium; (ii) stationing for about 1 minute at room temperature to obtain the supernatant; (iii) centrifuging the supernatant at about 1,500 rpm to obtain its cell pellet; (iv) resuspending the pellet in a new glia culture medium and seeding the solution in a plate for about 1 hour; (v) and replacing the medium with the glia culture medium and the neuron medium.

The present invention also comprises method for using the system of the present invention in substitution or support of in vivo models using the system of the present invention as an animal model. In an embodiment the system of the present invention is used as an animal model in studies of oxygen-glucose deprivation, ischemic stroke, inflammation, cell death, gliosis, ischemic stroke, encephalitis, encephalitis, virus infection, , Amyotrophic Lateral Sclerosis or neurodegenerative disease, such as Parkinson disease and Alzheimer's disease, vascular dementia, Alzheimer's Disease and/or Huntington's Disease.

### DESCRIPTION OF THE DRAWINGS

FIG. 1 shows fluorescent staining images of co-culture of glia cells and neurons obtained from rat neonatal brain in an embodiment of the GNCM composition of the present invention prepared by combining the components of Table 1. At 11 days in vitro, the mixed culture was dyed for the observation of cell morphology. FIG. 1A shows astrocyte cytoskeleton marked with glial fibrillary acidic protein (GFAP) and colored in green, neuron dendrites marked with microtubule-associated protein 2 (MAP2) and colored in red, as well as cell nuclei marked with 4′,6-diamidino-2-phenylindole (DAPI) and colored in blue. FIG. 1B shows astrocyte transporters marked with Glial Glutamate Transporter 1 (GLT1) and colored in green, neuron dendrites marked by MAP2 and colored in red, as well as cell nuclei marked with DAPI and colored in blue.

FIG. 2 shows fluorescent staining images of co-culture of glia cells and neurons obtained from mouse neonatal brain in an embodiment of the mGNCM composition of the present invention prepared by combining the components of Table 2. At 16 days in vitro, the mixed culture of mouse brain cells was dyed for the observation of cell morphology and neuron-glia interaction. FIG. 2A shows astrocyte cytoskeleton marked with GFAP and colored in green, and cell nuclei marked with DAPI and colored in blue. FIGS. 2B and 2C show neuron dendrites marked with MAP2 and colored in red, as well as cell nuclei marked with DAPI and colored in blue, in different scales. FIGS. 2D and 2E show astrocyte transporters marked with GLT1 and colored in green, as well as cell nuclei marked with DAPI and colored in blue, in different scales. FIGS. 2F and 2G show astrocyte transporters marked with GLT1 and colored in green, neuron dendrites marked with MAP2 and colored in red, as well as cell nuclei marked with DAPI and colored in blue, in different scales. The astrocytes of FIG. 2 exhibit long, thin morphology similar to that of astrocytes in vivo. Moreover, astrocyte transporters marked with GLT1 indicate the formation of thin astrocytic processes which enwrap neurons.

FIG. 3 shows fluorescent staining images indicating synapse formation of a mouse neonatal brain primary culture cultivated using an embodiment of the mGNCM composition of the present invention prepared by combining the components of Table 2. At 16 days in vitro, the mixed culture of mouse brain cells was dyed for the observation of cell morphology and neuron-glia interaction. FIG. 3A shows neuron dendrite post-synapses marked with PSD95 and colored in green, presynaptic axon terminals marked with Vesicular Glutamate Transporter 1 (vGLUT1) and colored in red, cell nuclei marked with DAPI and colored in blue, and the overlapping regions of post-synapses and pre-synapses colored in yellow. FIG. 3B shows neuron dendrite post-synapses marked with PSD95 and colored in green, presynaptic axon terminals marked with vGLUT1 and colored in red, cell nuclei marked with DAPI and colored in blue, and the overlapping regions of post-synapses and pre-synapses colored in yellow on a smaller scale compared with FIG. 3A. The closeness between pre-synapses and post-synapses indicate possible sites of synapse formation. FIG. 3C shows neuron dendrite post-synapses marked with PSD95 and colored in green, as well as cell nuclei marked with DAPI and colored in blue. FIG. 3D shows presynaptic axon terminals marked with vGLUT1 and colored in red, as well as cell nuclei marked with DAPI and colored in blue.

FIGS. 4A-D show fluorescent staining images of mouse primary neonatal brain cells cultivated using an embodiment of the mGNCM composition of the present invention at 14 DIV. FIG. 4A shows a glia-neuron co-culture at normoxia, with neuron dendrites marked with MAP2 and colored in red, as well as cell nuclei marked with DAPI and colored in blue. FIG. 4B shows a glia-neuron co-culture at normoxia, with microglia marked with Iba1 and colored in green, as well as cell nuclei marked with DAPI and colored in blue. FIG. 4C shows a glia-neuron co-culture subjected to oxygen-glucose deprivation (OGD), with neuron dendrites marked with MAP2 and colored in red, as well as cell nuclei marked with DAPI and colored in blue. FIG. 4D shows a glia-neuron co-culture subjected to OGD, with microglia marked with Iba1 and colored in green, as well as cell nuclei marked with DAPI and colored in blue. As can be seen, the number of neurons decreased following OGD treatment. In addition, microglia, cells responsible for immune responses in the brain, have transformed from thinner resting microglia to more hypertrophic activated microglia. FIGS. 4A-D illustrate that the glia-neuron co-culture system of the present invention also comprises microglia, and that the cells of said system exhibit immune reactions similar to that of brain cells in vivo.

FIGS. 4E and 4F show immunofluorescent images of the hippocampal CA1 and CA3 subregions under both sham and transient hypoxic-ischemia (tHI) conditions, with astrocytes marked with GFAP and colored in green and colored in green and microglia marked with Iba1 and colored in red. As can be seen, in both the CA1 and CA3, tHI insult induces astrocyte and microglial hypertrophy.

FIGS. 4G and 4H show immunofluorescent images of rat cingulate cortex, with Iba1 used as a marker for microglia, colored in green. As can be seen in the difference between the sham and chronic cerebral hypoperfusion (CCH) groups, CCH induces microglia proliferation and hypertrophy in rat cingulate cortex, similar to results of the in vitro model of the present invention.

FIGS. 41 and 4J are taken from Chen, W., Chang, L., Huang, S. et al. Aryl hydrocarbon receptor modulates stroke-induced astrogliosis and neurogenesis in the adult mouse brain. J Neuroinflammation 16, 187 (2019). https://doi.org/10.1186/s12974-019-1572-7 and hereby incorporated shows representative immunohistochemical staining for neuroinflammation at the peri-infarct cortex (bregma=+0.86 mm) of normal WT (n=6/each group), poststroke WT-Vehicle treated, WT-TMF treated, normal AHRcKO, poststroke AHR^{fix/fix}, and AHRcKO mice. The inset in low magnification is shown with a high magnification view in the next row(s). At high magnification, note the increase in AHR immunoreactivity colocalized within Iba1-positive microglia and GFAP-positive astrocytes (red arrows) in the WT-Vehicle-treated and AHR^{fix/fix} mice after MCAO, which was markedly decreased in the WT-TMF-treated and ARHcKO mice (whose AHR deletion was spared in microglia), respectively. Furthermore, increased Iba1-positive microgliosis and GFAP-positive astrogliosis in the WT-Vehicle and AHR^{fix/fix} mice after MCAO were significantly decreased in the WT-TMF-treated and AHRcKO mice (whose AHR was deleted in astroglia), respectively. Intracellular AHR immunoreactivity was predominantly in the nucleus, and to a lesser extent in the cytoplasm. The scale bars represent 60 μm at low magnification and 8 μm at high magnification.

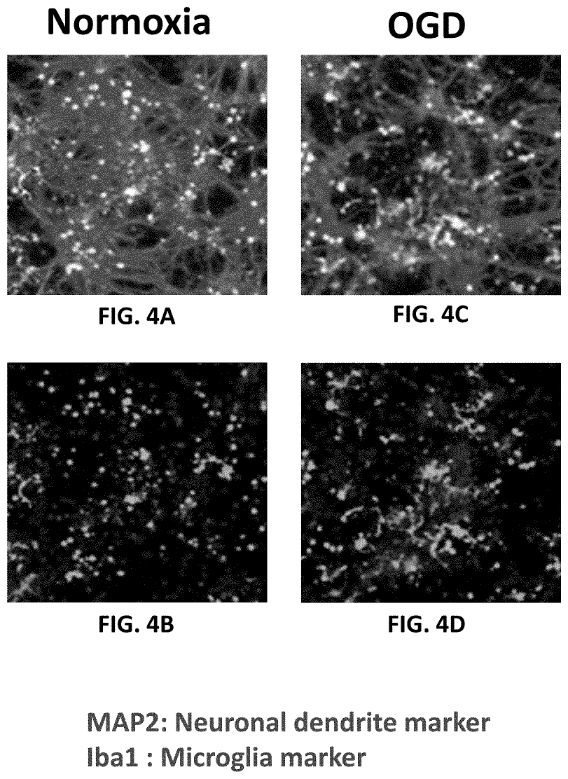

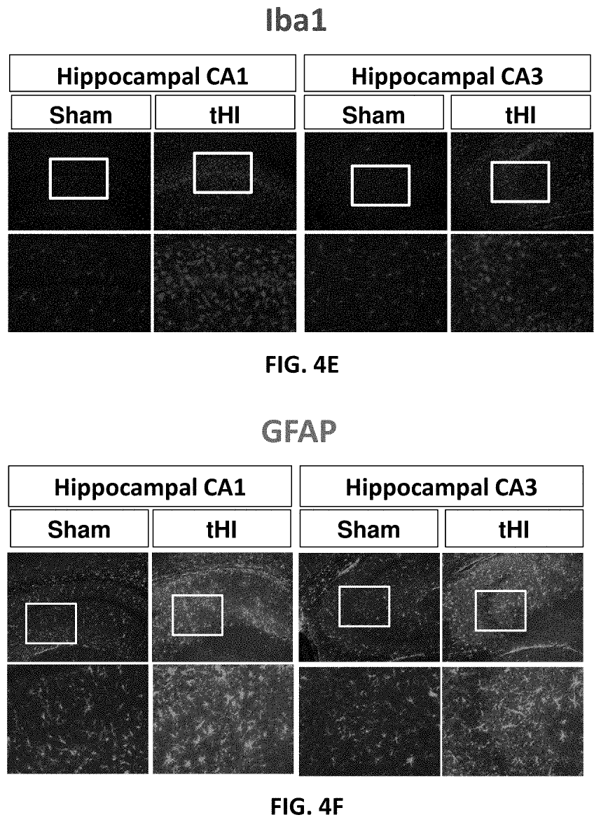

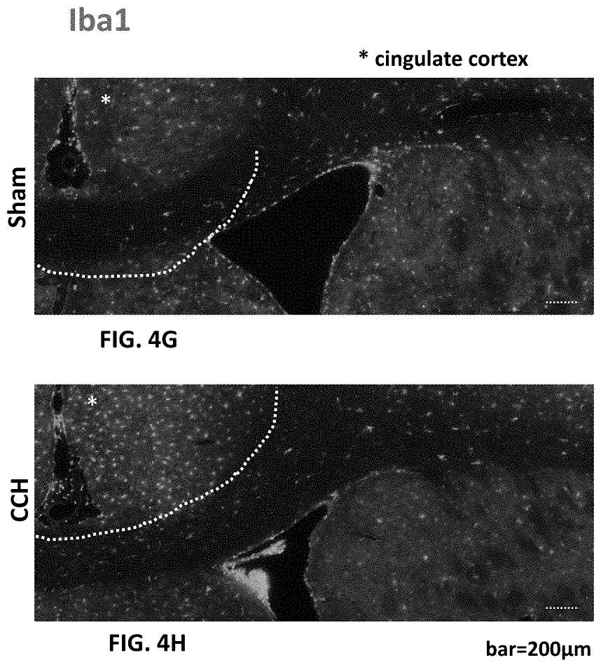

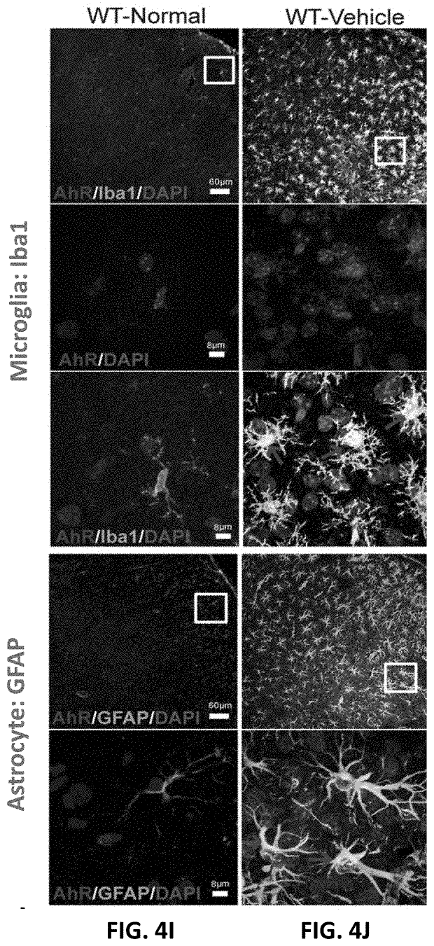

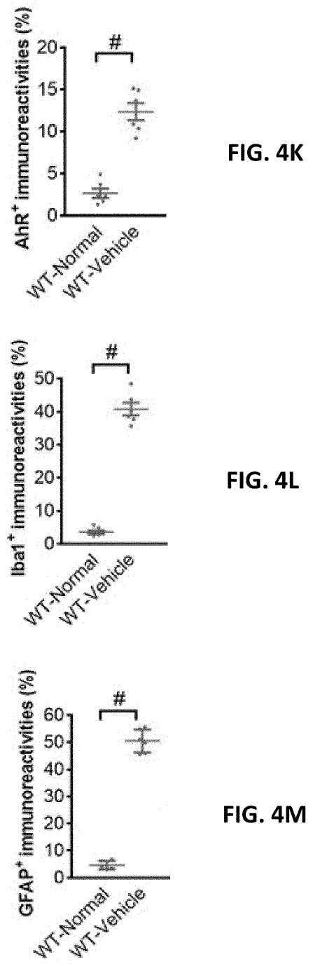

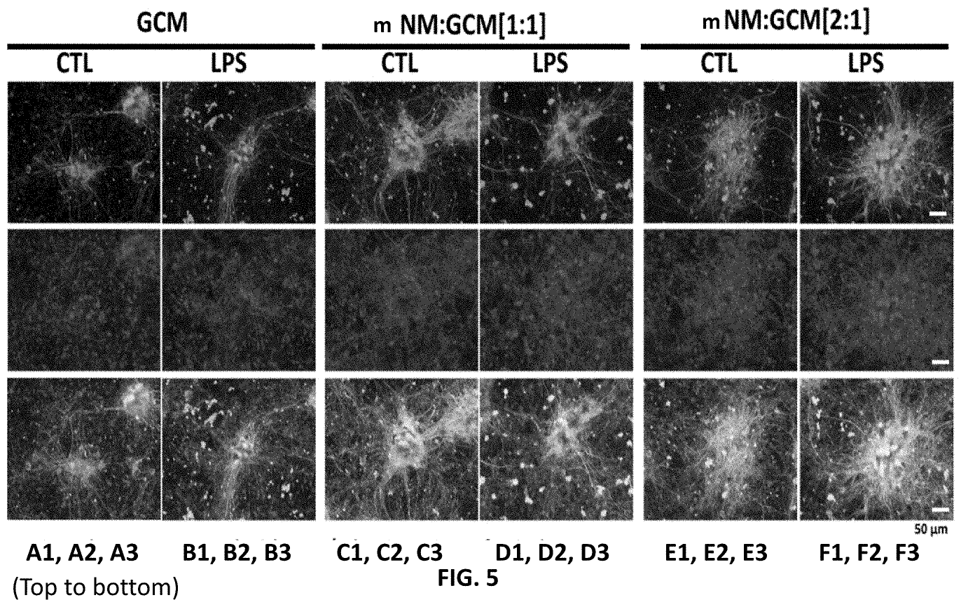

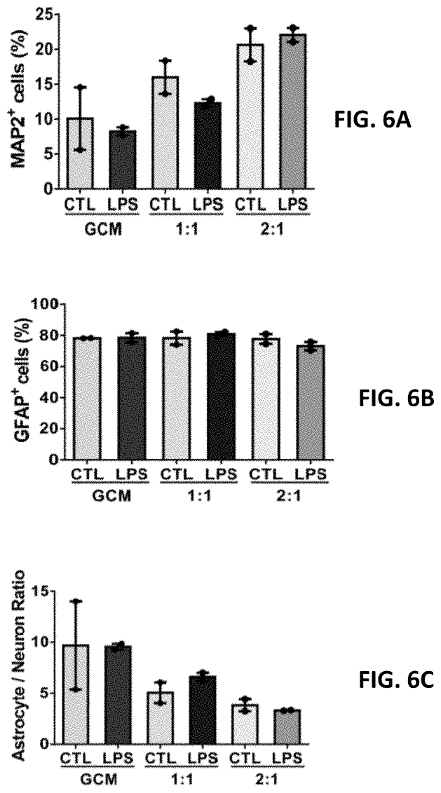

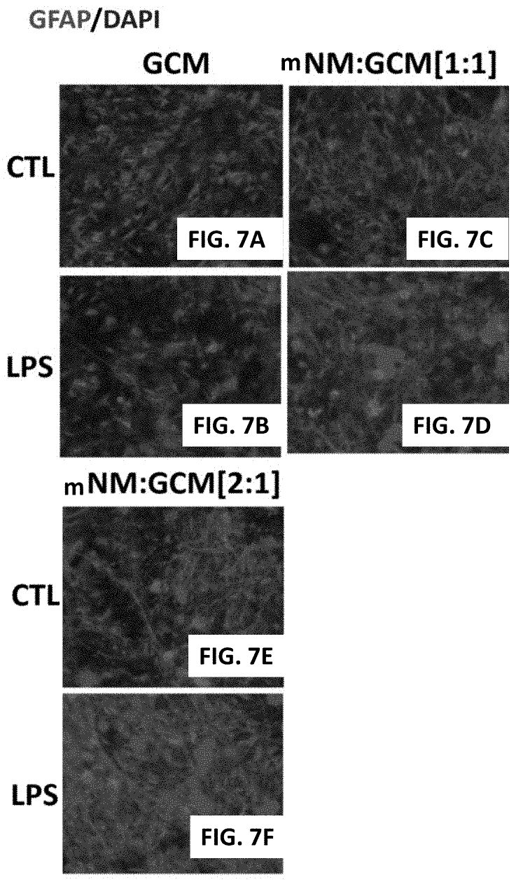

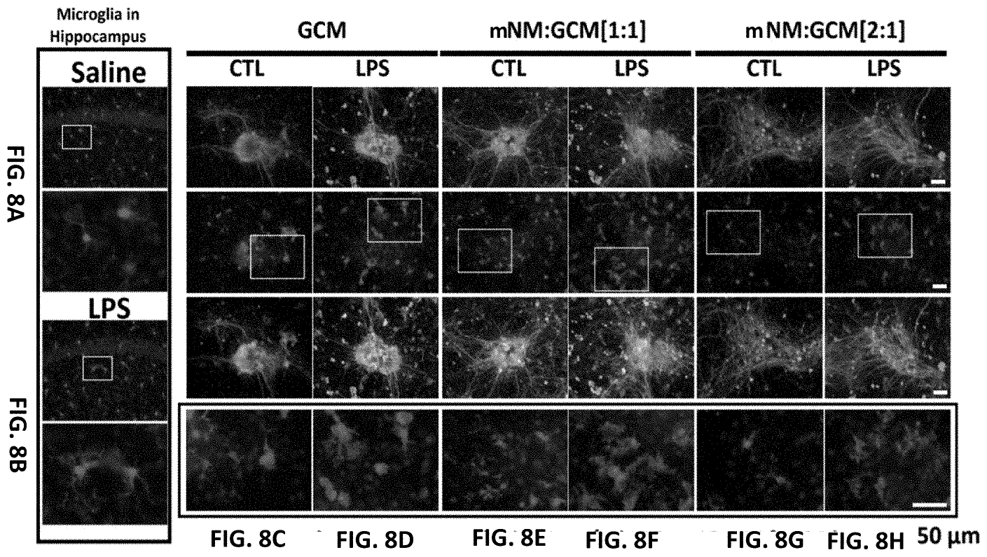

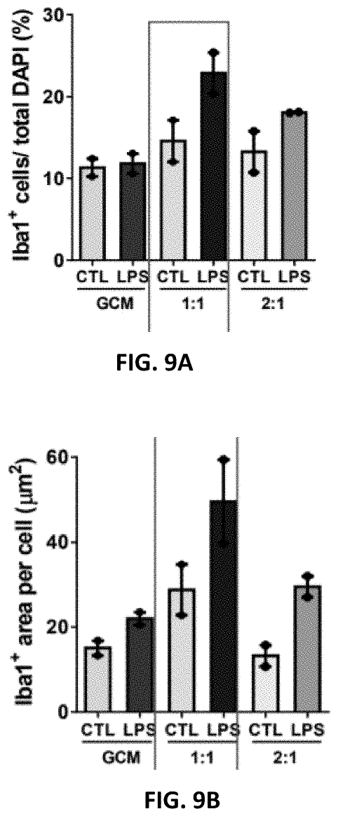

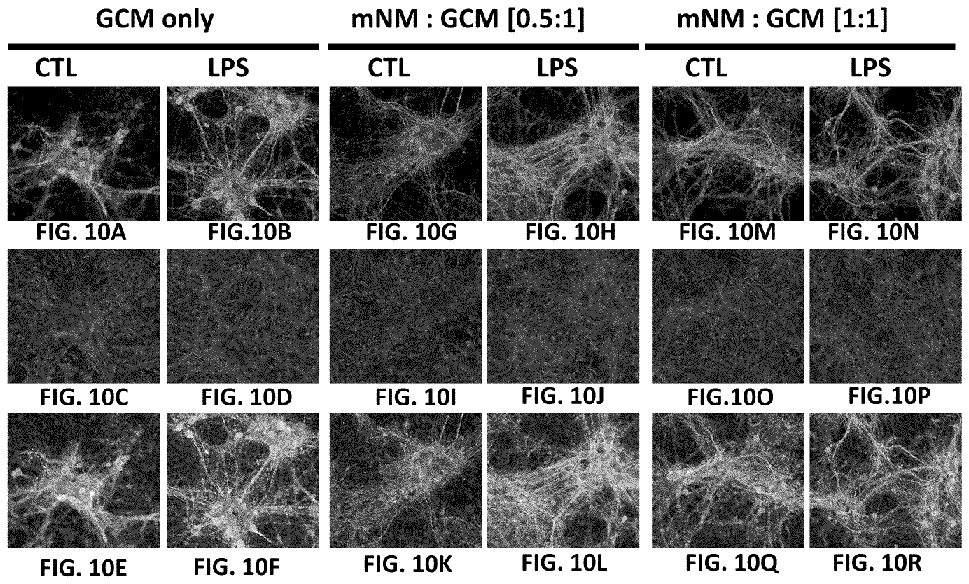

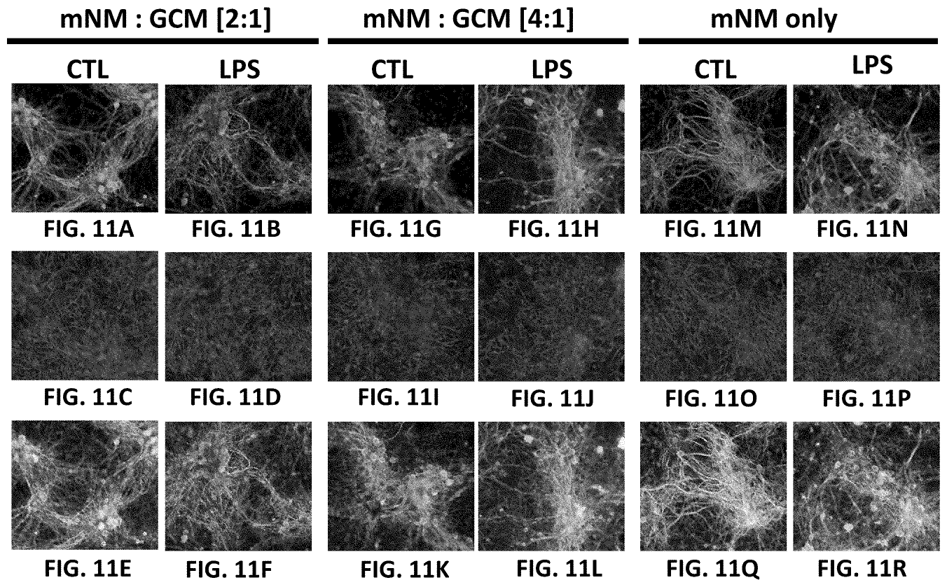

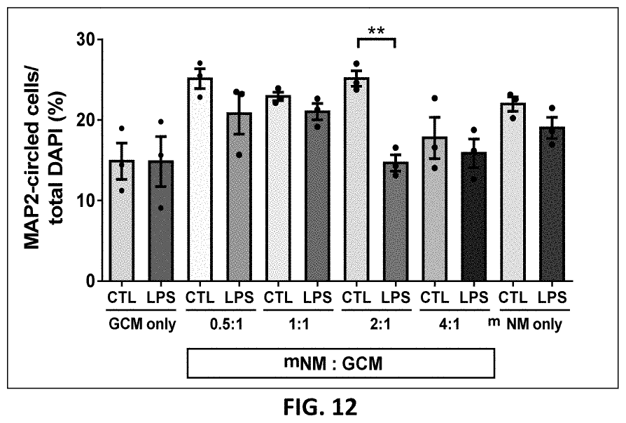

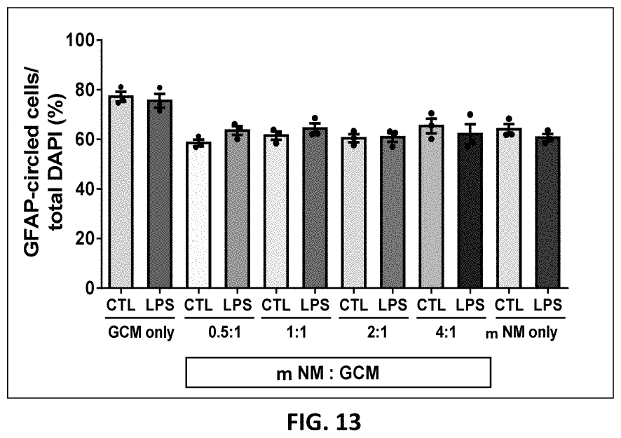

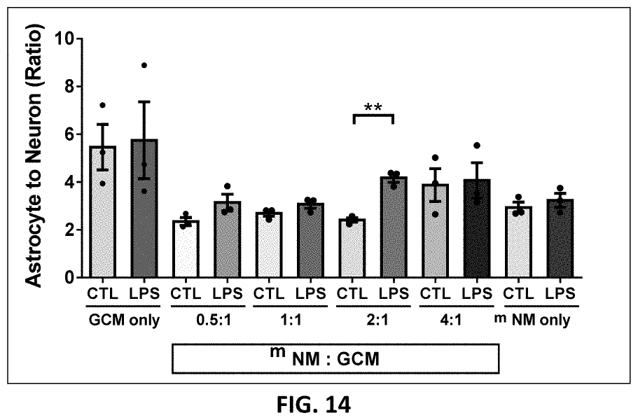

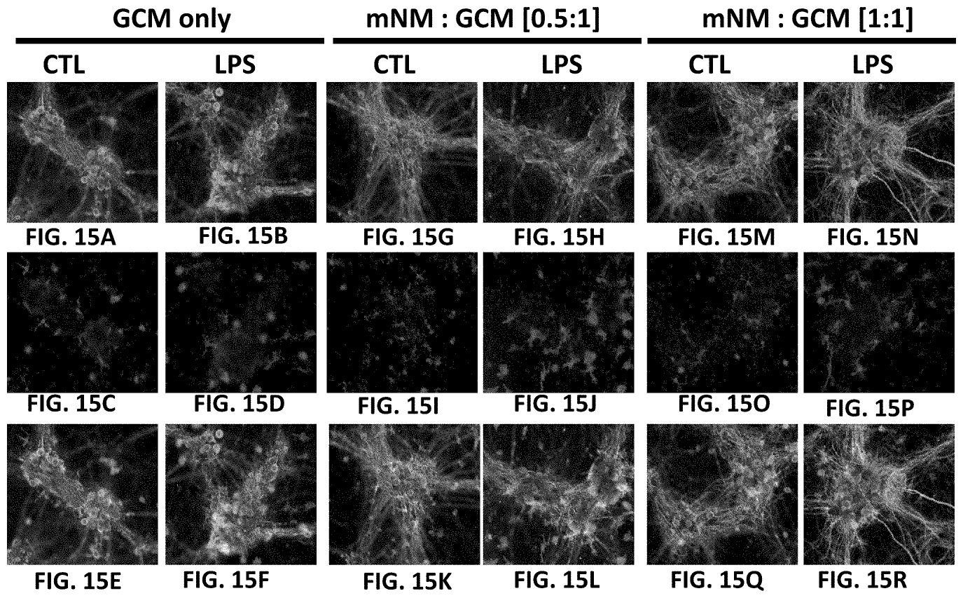

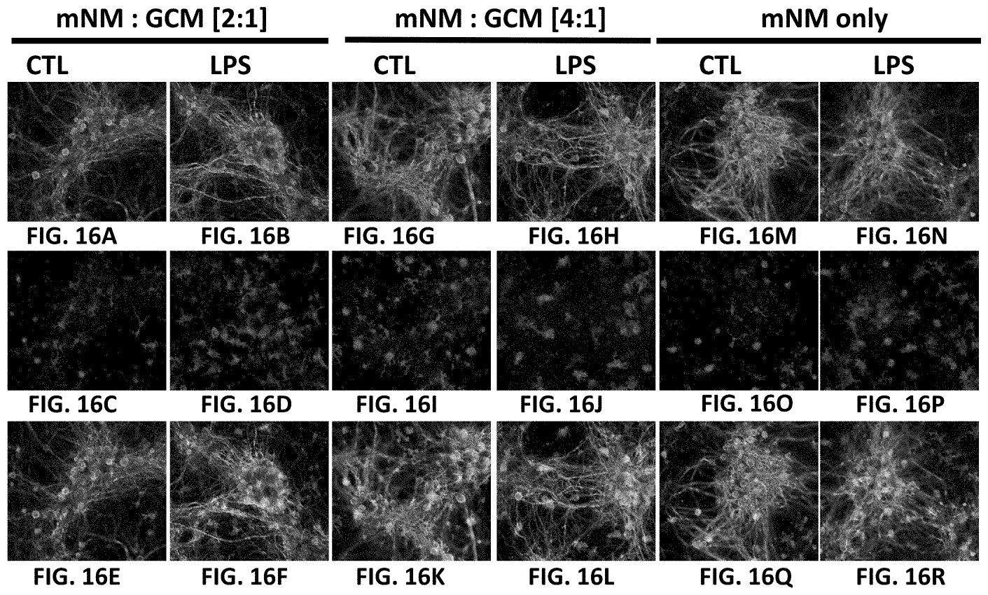

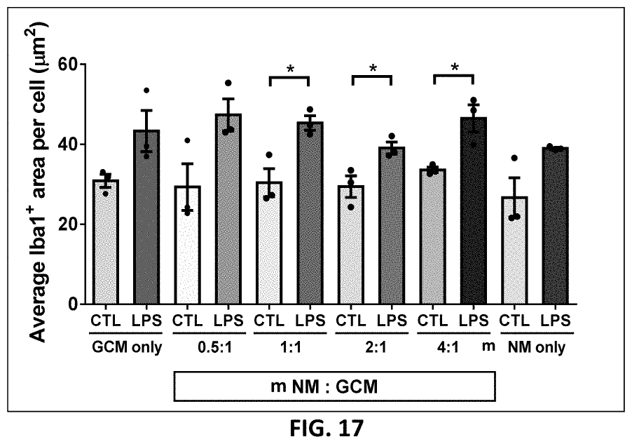

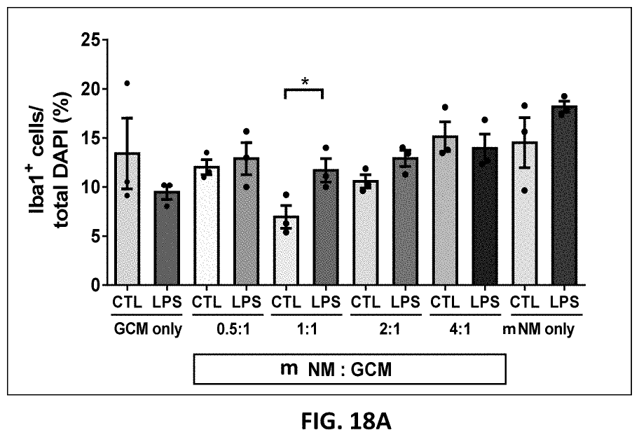

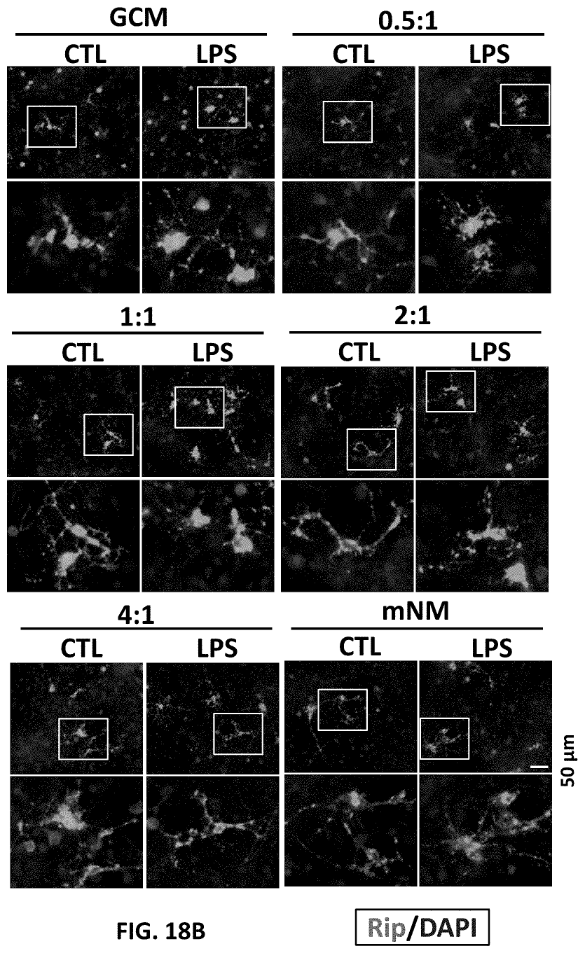

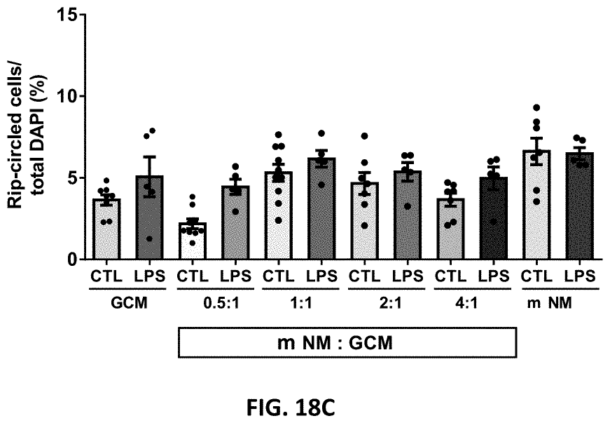

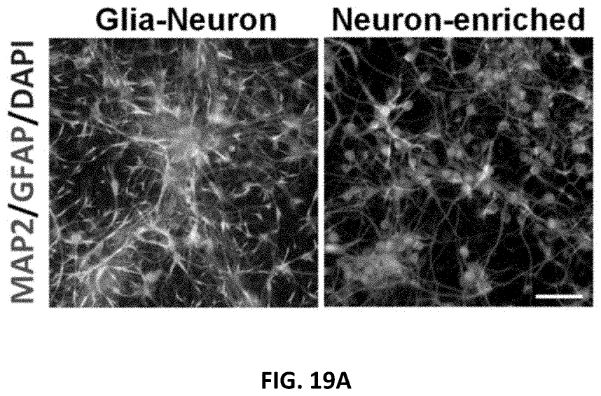

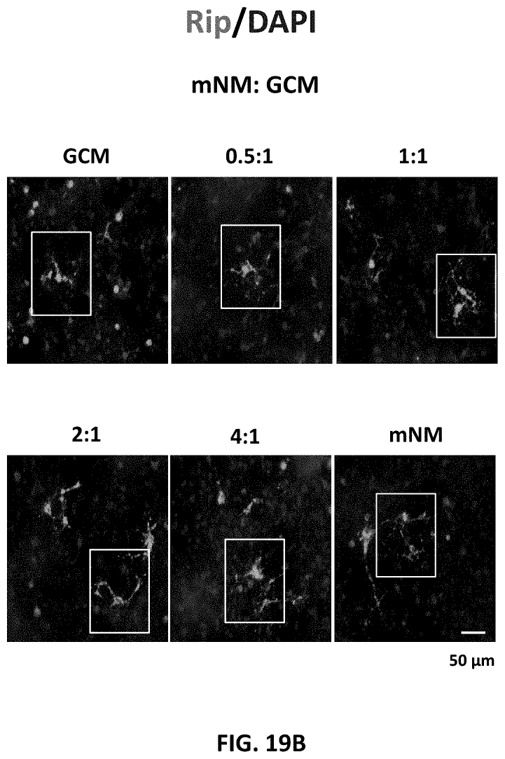

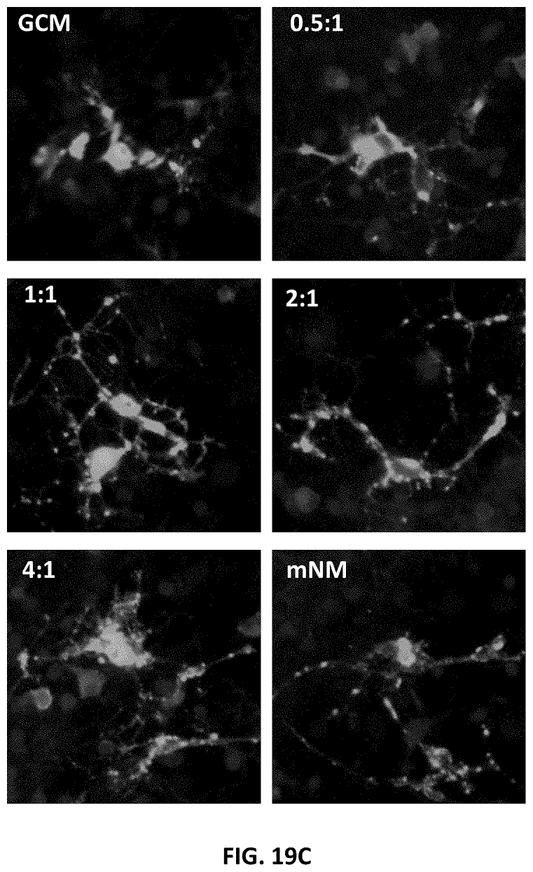

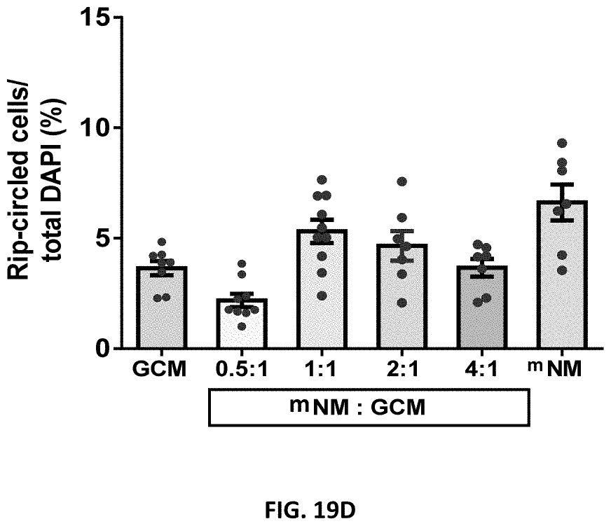

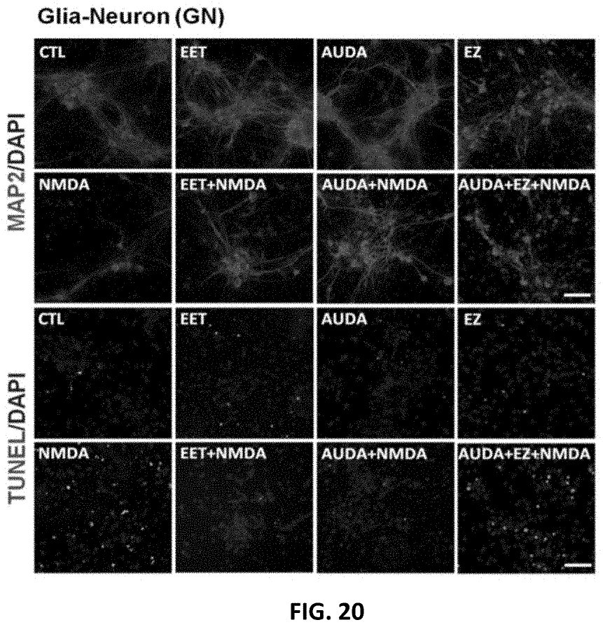

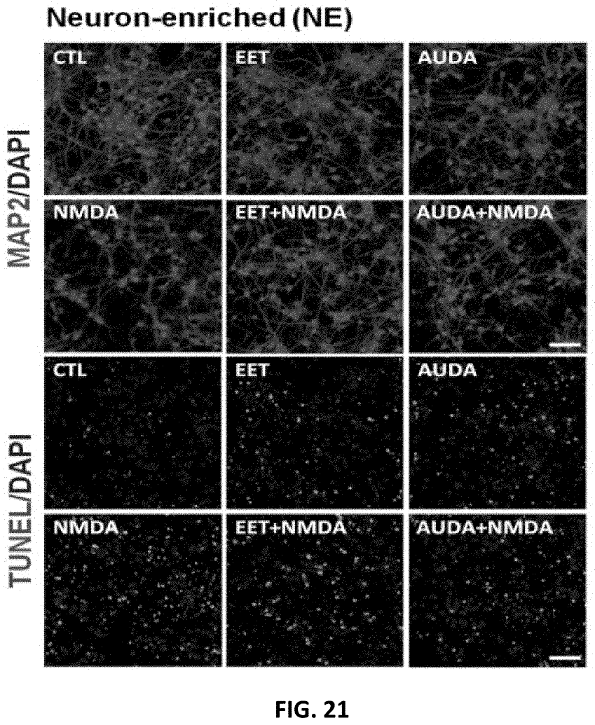

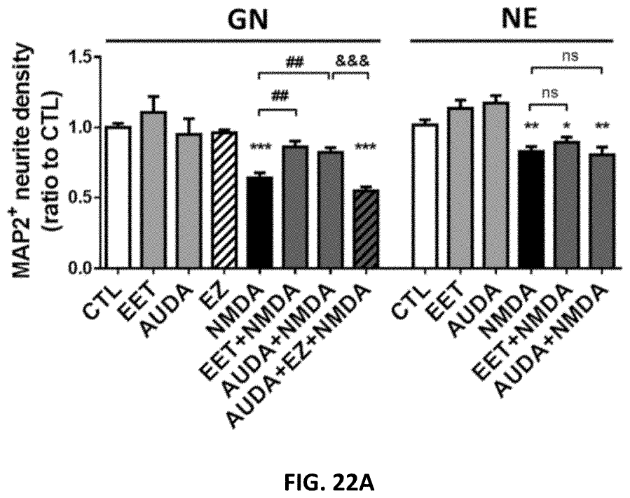

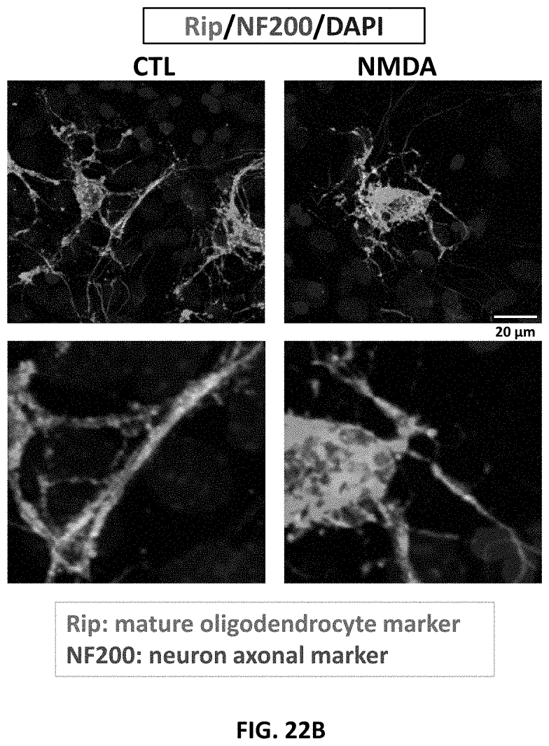

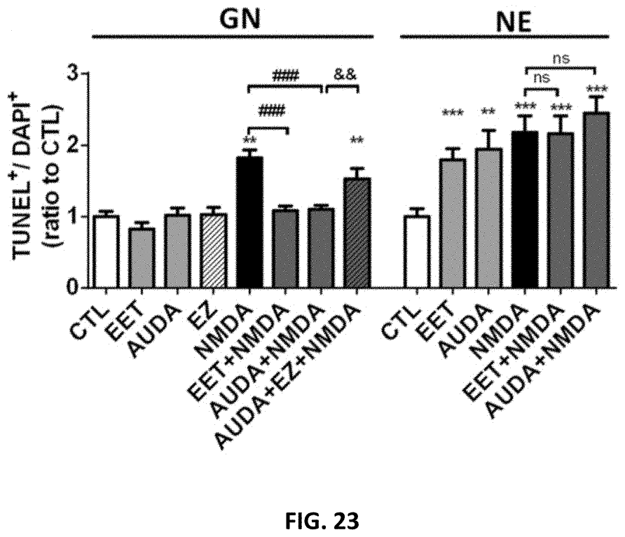

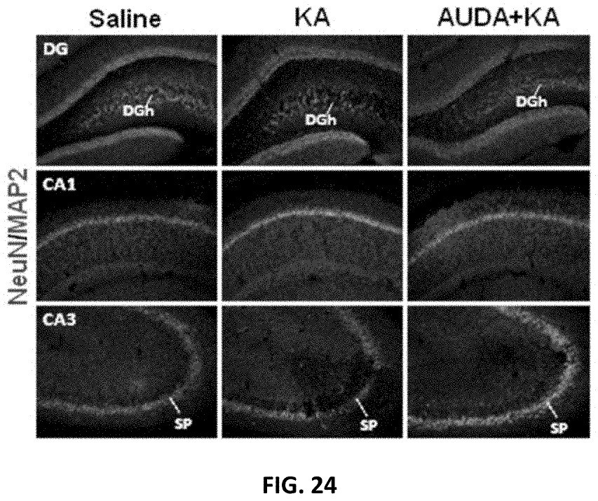

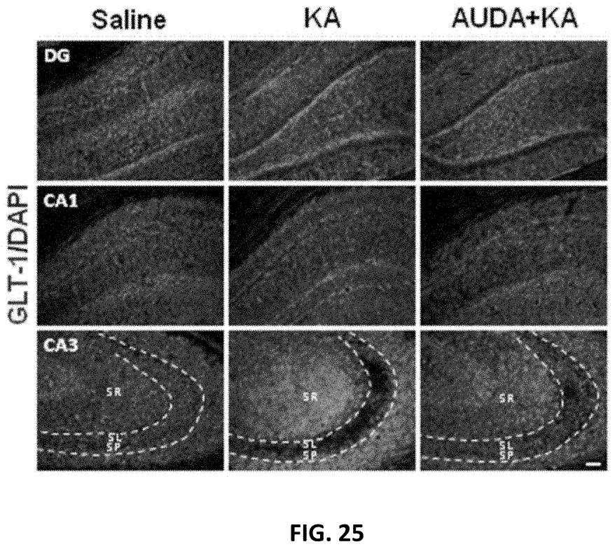

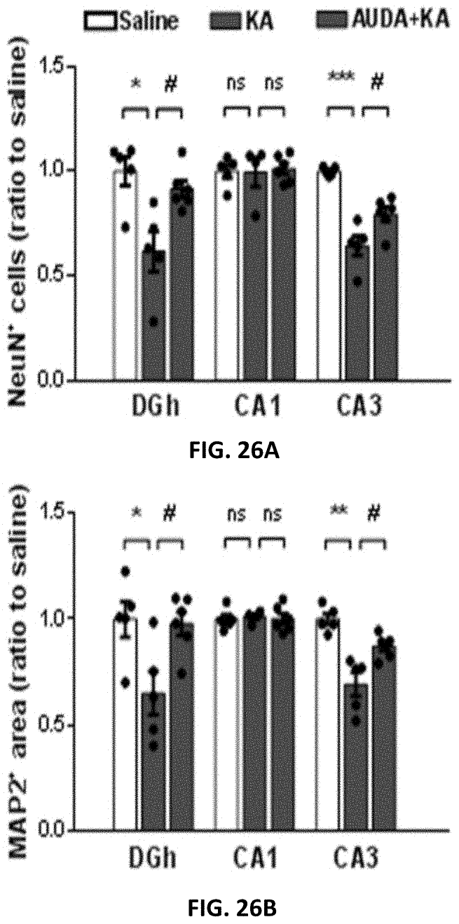

FIGS. 4K-L show quantifications of the relative immunereactive stained with AHR, Iba-1, and GFAP, respectively. As shown in FIGS. 4E-4H, microglial and astroglial activation are results of MCAO in mice, similar to the results of FIG. 4A-D.

FIG. 5 shows effects of neuroinflammation simulation of rat cerebral cortex cells grown in vitro using the three culture media of Table 3. Cells obtained from rat cerebral cortex were cultured in vitro for 13 days using the three culture media, then treated with 1 μg/mL of lipopolysaccharides (LPS) for 24 hours on day 13 to trigger inflammation. Following LPS treatment, immunostaining was performed to observe cell population, morphology, and response to inflammatory stimulus. The immunostaining images of FIG. 5 show neuronal population and response to LPS stimulation, wherein CTL represents control groups (not subjected to LPS treatment) and LPS represents groups treated with LPS to induce neuroinflammation. The images show astrocyte cytoskeleton marked with GFAP and colored in red, neuron dendrites marked with MAP2 and colored in green, as well as cell nuclei marked with DAPI and colored in blue. FIGS. 5A1-5A3 and 5B1-5B3 show cells cultured with GCM only, FIGS. 5C1-5C3 and 5D1-5D3 show cells cultured with 1:1 NM and GCM, while FIGS. 5E1-5E3 and 5F1-5F3 show cells cultured with 2:1 NM and GCM. The GCM only group comprises fewer neurons, and the cells are smaller and unhealthier in both CTL and LPS treatment groups. Of the 1:1 group, neurons of the CTL group exhibit the morphology of healthy neurons, while signs of neuron damage and some cell debris in staining can be found in the LPS treatment group, indicating neuroinflammation. Of the 2:1 group, neurons of the CTL group exhibit the morphology of healthy neurons and are surrounded by more astrocytes while being less clustered. Though the number of neurons in the LPS treatment group does not seem to have decreased, the images show possible fiber sprouting, which may have been caused by inflammation.

FIG. 6 illustrates percentage by cell type of FIG. 5. FIG. 6A graphs the percentages of MAP2 positive cells, FIG. 6B graphs the percentages of GFAP positive cells, while FIG. 6C graphs the astrocyte/neuron ratios of each group. As shown in FIG. 6A, while LPS stimulation induces neuronal loss in the 1:1 group, the GCM only and 2:1 groups are not affected in this way. As such, 1:1 GNCM culture conditions are ideal for the study of inflammation-induced neuro toxicity and neurodegeneration. As can be seen in FIG. 6C, astrocyte/neuron ratios correlate reversely to the proportion of NM in the culture medium. Notably, the astrocyte/neuron ratios of the 1:1 group and the GCM only group, about 5:1 and about 10:1, respectively, lie within normal ranges found in the brains of rats.

FIG. 7 shows the morphological differences of astrocytes of different groups cultured with the cell culture media of Table 3, with astrocyte cytoskeleton marked with GFAP and colored in red, as well as cell nuclei marked with DAPI and colored in blue. Astrocyte hypertrophy induced by LPS stimulation can be found in both the 1:1 group and 2:1 group, as shown in FIGS. 7D and 7F, but the same effect is not observed in the GCM only group, as shown in FIG. 7B. Moreover, inflammation-induced astrogliosis is more pronounced in the 1:1 and 2:1 groups, making these groups good approximations of neuroinflammation of brain cells grown in vivo. Notably, though the GCM only group comprises more glia cells, its response to inflammatory stimulus is inferior to that of the 1:1 and 2:1 groups, as shown in FIGS. 7A and 7B.

FIG. 8 compares the density and morphology of microglia in the hippocampus of rats and in cell culture media of Table 3, wherein neuron dendrites are marked with MAP2 and colored in green, microglia are marked with Iba1 and colored in red, and cell nuclei are marked with DAPI and colored in blue. As shown in FIG. 8E, the microglial cells of the 1:1 group exhibit surveillance-like thin processes morphology similar to those of the hippocampus saline group, shown in FIG. 8A as disclosed in Neuron. 2018 Jan 17; 97(2): 299-312.e6. doi: 10.1016/j.neuron.2017.12.002. Moreover, as shown in FIG. 8F, the reaction of microglia to LPS of the 1:1 group show marked increase in both cell density and cell size, more similar to that of the hippocampus in LPS-injected mouse, shown in FIG. 8B. Thus, cells cultured with the 1:1 NM and GCM medium are well suited to simulation of cells cultured in vivo, as well as to simulation of morphology and behavior of microglia grown in vivo. FIG. 8G shows that microglia of the 2:1 group exhibit unhealthier morphology as compared with those of the hippocampus and 1:1 groups. Though the reaction of the 2:1 group to LPS stimulus, shown in FIG. 8H, appears to be more consistent, it may be a result of its unhealthier baseline condition.

FIG. 9 quantifies the results of fluorescent imaging of FIG. 8, with FIG. 9A graphing the percentage of Iba1 positive cells in the total count of DAPI stains, namely the percentage of microglia in total cells, and FIG. 9B showing the Iba1 positive area per cell. As can be seen, the 1:1 group is superior on both counts compared to the GCM only and 2:1 groups.

To examine cell type population and morphology of rat glia cell and neuron mixed culture, six cell culture media were prepared to the specifications of Table 4. The neuron medium of the present embodiment comprises Neurobasal™ medium containing 4.5 g/L glucose, and additional 0.75 g/L glucose. The glia culture medium of the present embodiment comprises DMEM containing 4.5 g/L glucose, lx penicillin-streptomycin, and 10% FBS.

On postnatal day 0 (P0), cells extracted from rat cerebral cortex were seeded in each of the cell culture media of Table 4. Following 14 days of in vitro cultivation, immunostaining was performed on the control groups (CTL) to aid in the observation of cell type ratios, specifically of neurons, astrocytes, microglia and oligodendrocytes as well as cell morphology. Cultures of the LPS groups (LPS) were subjected to inflammatory stimulation via 1 μg/mL LPS at 13 days in vitro. After another 24 hours, the LPS groups were subjected to immunostaining as well, for the observation of cell morphology and cell count.

FIGS. 10 and 11 show immunostaining results of co-culture systems of different proportions of mNM and GCM of the present invention prepared to the specifications of Table 4, including both control and LPS groups of cells cultivated in GCM only medium, mNM:GCM 0.5:1 medium, mNM:GCM 1:1 medium, mNM:GCM 2:1 medium, mNM:GCM 4:1 medium, and mNM only medium. The neuron medium of the present embodiment comprises Neurobasal™ medium containing 4.5 g/L glucose, and additional 0.75 g/L glucose. The glia culture medium of the present embodiment comprises DMEM containing 4.5 g/L glucose, lx penicillin-streptomycin, and 10% FBS. On postnatal day 0 (P0), cells extracted from rat cerebral cortex were inoculated in each of the cell culture media of Table 4. Following 14 days of in vitro cultivation, immunostaining was performed on the control groups (CTL) to aid in the observation of cell type ratios, specifically of neurons, astrocytes, and microglia, as well as cell morphology. Cultures of the LPS groups (LPS) were subjected to inflammatory stimulation via 1 μg/mL LPS at 13 days in vitro. After another 24 hours, the LPS groups were subjected to immunostaining as well, for the observation of cell morphology and cell count. The images show astrocyte cytoskeleton marked with GFAP and colored in red, neuron dendrites marked with MAP2 and colored in green, as well as cell nuclei marked with DAPI and colored in blue. As can be seen, LPS stimulation induces astrocyte hypertrophy and process arborization. In addition, the addition of NM promotes the formation of thinner astrocyte processes. As seen in FIGS. 10A-10F, co-cultures grown in GCM only medium produce fewer neurons, and that the neuronal processes are unnaturally fat and short. As astrocyte morphology is closely linked to that of neurons, the astrocytes of the GCM only group exhibit abnormal morphology. Some reaction to LPS may be seen, likely the result of glia response. Co-cultures grown in mNM:GCM 0.5:1 medium, shown in FIGS. 10G-10L, exhibit improved cell morphology over the GCM only group, though neuronal processes are still shorter than their in vivo state. LPS treatment induces neuron outgrowth in this group, and unhealthy neurite stress fibers similar to the morphology of more aged cell cultures are observed. As FIGS. 10M-10R show, neuron morphology is more relaxed and natural when the co-culture is cultivated in mNM:GCM 1:1 medium. The co-cultures of the GCM only group, the 0.5:1 group, and the 1:1 group all react to LPS, though neurons are more impacted, and some induced neuroplasticity can be observed.

FIGS. 11A-11F show the immunostaining results of the mNM:GCM 2:1 group. Some neuron damage can be observed following LPS treatment. FIGS. 11G, 11I, and 11K show that neurons cultured in mNM:GCM 4:1 medium without LPS treatment exhibit unhealthy morphology. Some response to LPS treatment can be seen in FIGS. 11H, 11J, and 11L. In the mNM only group, shown in FIGS. 11M-11R, neurons form clusters and fewer processes, unlike their natural, in vivo, state.

FIG. 12 shows the percentage of MAP2-circled cells in total DAPI, meaning the percentage of neurons in total cells, for each of the groups of Table 4. Interestingly, though the presence of mNM increases neuron percentage, higher ratios of mNM in the co-culture media don't necessarily produce larger neuron populations, likely because neurons do not proliferate, and that their cell count depends on initial seeding. As can be seen, LPS treatment reduced the percentage of neurons in the co-culture systems of all groups except for the GCM only group. This could be a combined result of cell death due to inflammatory neurotoxicity and glia proliferation. In addition, significant neuronal loss following LPS treatment may be observed in the 2:1 mGNCM group, indicating that the 2:1 medium is useful in simulating LPS-induced neurotoxicity and neurodegeneration that can be observed in vivo. Therefore, the mNM:GCM 2:1 co-culture system may be used as a screening platform for neurodegeneration caused by infectious diseases.

FIG. 13 shows the percentage of GFAP-circled cells in total DAPI, meaning the percentage of astrocytes in total cells, of each of the groups of Table 4. As can be seen, LPS treatment does not cause significant proliferation or death of astrocytes, indicating that the decrease in neuron percentage observed in FIG. 12 is mainly a result of neuronal death.

FIG. 14 shows the ratio of astrocytes to neurons of each of the groups of Table 4. As can be seen, the astrocyte to neuron ratios range from about 2:1 to about 6:1, which fall within the range that can be found in vivo. Notably, in the 2:1 group, there is a significant difference of astrocyte to neuron ratio between the control group and LPS group, indicating significant neurodegeneration, as stated above.

FIGS. 15 and 16 show immunostaining results of both control and LPS groups of cells cultivated in GCM only medium, mNM:GCM 0.5:1 medium, mNM:GCM 1:1 medium, mNM:GCM 2:1 medium, mNM:GCM 4:1 medium, and mNM only medium, prepared to the specifications of Table 4. The images show microglia marked with Iba1 and colored in red, neuron dendrites marked with MAP2 and colored in green, as well as cell nuclei marked with DAPI and colored in blue. As can be seen, LPS stimulation induces microglial hypertrophy (microgliosis) in all groups except for the GCM only group, in which the microglia in resting state, namely those of the control group, are already overly active. Also notable is the fact that resting microglia of the mNM only group are a round, unhealthy shape, different from that of their natural, in vivo morphology. FIGS. 16A-16F show that in the mNM:GCM 2:1 co-culture system, LPS induces an increase of microglia, indicating microglial proliferation or recruitment. FIGS. 16G-16L show that microglia of the control group are more active, indicating that the neurons are sickly, and that the reaction to LPS treatment is diminished as compared to the 0.5:1, 1:1, and 2:1 groups.

FIG. 17 shows averaged Iba1 absolute area per cell, meaning the averaged size of microglial cells per culture area, of each group of Table 4. As noted above, microglia morphology of the 1:1, 2:1, and 4:1 groups show hypertrophy under LPS stimulation, the results of which are confirmed in FIG. 17 by significant differences of Iba1 positive areas per cell between the control groups and LPS groups.

FIG. 18A shows the percentage of Iba1 positive cells over in total DAPI, meaning the percentage of microglia in total cells, of each group of Table 4. As can be seen, there is a significant difference in microglia population between the control and LPS groups of the 1:1 group, indicating that LPS induces significant microglia proliferation only in mNM:GCM 1:1 medium. In addition, microglial population in all medium conditions are in the range of 7-15%, similar to the population in the brain.

FIGS. 18B and 18C illustrate morphological plasticity of oligodendrocyte in response to 1 μg/mL LPS treatment in GN mix culture. Oligodendrocytes were marked with RIP (mature oligodendrocyte marker). Oligodendrocyte forms the myelin sheath to enwrapping mature neuronal axon to improve neuronal transduction As the figures illustrate, LPS does not affect oligodendrocyte (OL) cell density in culture, but induces the oligodendrocyte process shrinkage in mNM: GCM=0.5:1 and 1:1 media, which can be applied to study an inflammation-induced oligodendrocyte damage in demyelinating disease.

FIG. 19A shows the resulting immunofluorescent images of 9-10 DIV cultures of neuron-enriched (NE) cell culture as well as a GN culture, in which neurons were characterized by immunolabeling with MAP2 shown in red, while astrocytes were characterized by immunolabeling with GFAP shown in green, and cell nuclei were marked with DAPI and shown in blue.

FIGS. 19B and 19C are images of oligodendrocytes in rat GN mix culture where oligodendrocytes were marked with RIP (mature oligodendrocyte marker) and colored in green and cell nuclei were marked with DAPI and colored in blue. As shown in the figures, oligodendrocyte morphology in mNM:GCM=1:1, 2:1, and 4:1 medium appears to be most similar to oligodendrocyte in the brain (image data).

FIG. 19D summarizes the oligodendrocyte population: mNM:GCM=1:1, 2:1, 4:1 and NM medium yield from 3.5 to 6.5% oligodendrocyte.

FIG. 20 shows immunostaining images of the GN culture, including the control group, and groups subjected to 14,15-EET, sEH inhibitor AUDA, EZ, NMDA, 14,15-EET and NMDA, AUDA and NMDA, and AUDA, EZ, and NMDA treatment. Neuron dendrites were marked with MAP2 and colored in red, cell nuclei were marked with DAPI and colored in blue, while cell death was marked with TUNEL and colored in green. Scale bar=50 μm.

FIG. 21 shows immunostaining images of the NE culture, including the control group, and groups subjected to 14,15-EET, AUDA,NMDA, 14,15-EET and NMDA, as well as AUDA and NMDA treatment. Neuron dendrites were marked with MAP2 and colored in red, cell nuclei were marked with DAPI and colored in blue, while cell death was marked with TUNEL and colored in green. Scale bar=50 μm.

FIG. 22A shows quantification of MAP2⁺ neurite density in FIGS. 20 and 21.

FIG. 22B illustrate myelination of neuronal axons by oligodendrocytes in GN mix culture, and in response to excitotoxic NMDA treatment that mimic excitotoxicity-induced demyelination. Neural axons were marked with NF200 and colored in red, oligodendrocytes were marked with RIP and colored in green and cell nuclei were marked with DAPI and colored in blue. As seen in the figures, oligodendrocyte morphology in NM:GCM=1:1 medium for 14 DIV reveals process branching and forms myelinsheath enwrapping the axon, but glutamate receptor agonist NMDA-induced excitotoxicity disrupts oligodendrocyte process extension and arborization.

FIG. 23 shows quantification of TUNEL⁺ cells in FIGS. 20 and 21. Both MAP2⁺ neurite density and TUNEL⁺ cells were normalized by the total DAPI number in each quantified microscopic field. Note that NMDA-induced MAP2⁺ neurite reduction and cell death are significantly attenuated by both 14,15-EET and AUDA treatments in the GN cultures, but not in NE cultures. Results indicate that 14,15-EET and sEH inhibitor treatments attenuated NMDA-induced neurotoxicity and hyperexcitation in GN mix but not in NE cultures.

FIG. 24 shows representative immunofluorescent images of different hippocampal areas double stained with neuronal nuclei marker NeuN (green) and dendritic marker MAP2 (red), following unilateral i.c.v. injection of saline, KA, or KA with the sEH inhibitor AUDA.

FIG. 25 shows representative immunofluorescent images of different hippocampal areas with GLT-1 staining (green) counterstained with DAPI (blue), following unilateral i.c.v. injection of saline, KA, or KA with the sEH inhibitor AUDA. Notably, excitotoxicity induced neuronal loss in the stratum pyramidale (SP) and astrocyte hypertrophy in the stratum raditum (SR) in rodent hippocampal CA3.

FIG. 26 shows quantitative analyses of images in FIG. 24. NeuN⁺ cells of each hippocampal subregion were determined by counting the number of NeuN⁺/DAPI⁺ cells and then normalized with respective saline treatment group. MAP2 area was determined by the percentage of the immunoreactive area in each hippocampal region of interest and then normalized with the respective saline treatment group. Note that AUDA treatment significantly attenuated the KA-induced NeuN⁺ cell loss and MAP2⁺ dendrite damage in DGh and CA3. Scale bar=100 μm.

FIG. 27 is a quantitative analysis of images in FIG. 25. GLT-1 area was determined by the percentage of the immunoreactive area in each hippocampal region of interest and then normalized with the respective saline treatment group. Note that GLT-1 immunoreactivity was depleted in the stratum pyramidale (SP) and stratum lucidum (SL) layers of CA3 in the KA treatment group, and the effect was attenuated by the AUDA pretreatment.

FIG. 28 shows immunofluorescent images of CA1 and CA3 subregions of the hippocampus, injected with saline or LPS. Astrocytes were marked with GFAP and colored in green, while cell nuclei were marked with DAPI and colored in blue. As can be seen, LPS induced astrocyte hypertrophy and proliferation in mouse hippocampal CA1 and CA3, similar to the in vitro results of FIGS. 7, 10, and 11.

FIG. 29 shows immunofluorescent images of the CA1 subregion of the hippocampus, injected with saline or LPS. Microglia were marked with Iba-1 and colored in green, while cell nuclei were marked with DAPI and colored in blue. As can be seen, LPS induced microglial hypertrophy and proliferation in mouse hippocampal CA1, similar to the in vitro results of FIGS. 15, 16, and 17.

### DETAILED DESCRIPTION OF THE INVENTION

As used in this specification and in claims which follow, the singular forms “a”, “an,” and “the” include plural referents unless the context clearly indicates otherwise.

As used herein, the term “about” as a modifier to a quantity is intended to mean + or −5% inclusive of the quantity being modified.

The glia cell and neuron co-culture system of the present invention comprises glia cell and neuron co-cultured within glia-neuron culture medium (GNCM) as defined in further details below. In another embodiment, the glia cell and neuron co-culture system of the present invention comprises glia cells and neurons co-cultured within modified glia-neuron culture medium (mGNCM) as defined in further details below. The co-culturing glia cells and neurons of the present invention means more than merely combining glia and neuron cells together in the same culture but also involves culturing the glia cells and neurons together so that the two types of cells can mature in the presence of each other for at least 1 day, 2 days, 3 days, or 4 days or 5 days etc. . . . up to 21 days, 30 days, 40 days or 45 days.

Both GNCM and mGNCM each comprises a mixture of DMEM and Neurobasal™ for co-culturing glia cells and neurons that provides cell morphology, cell reaction (such as apoptosis, immunological/inflammatory response, neurogenesis, gliosis, etc. . . . ) and/or cell interactions, including those described in the Examples below, that are closely aligned with those in vivo. These cell morphology, cell reaction and/or cell interactions have not yet been seen in existing systems and method for in vitro culturing glia and neuron cells prior to the present invention. These advantages allows for the system and method of the present invention to be configured for use as animal and human cell modeling for research, drug screening, tests and/or clinical trials related to such areas of study as oxygen-glucose deprivation, ischemic stroke, inflammation, cell death, gliosis, ischemic stroke, encephalitis, and neurodegenerative disease, such as Parkinson disease and Alzheimer's disease, Huntington's disease, multiple sclerosis, amyotrophic lateral sclerosis, vascular dementia etc. . . .

The system and method of the present invention is suitable, but not limited, to culturing glia and neuron cells obtained from brains of mammals such as mice, rats, dogs, non-human primates, sheep, cows or human beings. In another embodiment, the system and method of the present invention is suitable, but not limited, to culturing glia and neuron cells obtained from the neonatal brain primary cultures of rats or mice. Another embodiment of the present invention comprises the usage of the co-culture media of the present invention in cultivation of iPSc-derived neuron-glia mix cultures.

In an embodiment of the present invention, the co-cultured glia and neuron cells comprise cells normally existing in mammal brains at percentage that normally exists in mammal brains. In an embodiment of the present invention, the co-cultured glia and neuron cells comprise cells astrocytes and neuron cells. In another embodiment of the present invention, the glia cells comprise astrocyte, microglia, oligodendrocyte cells or a combination thereof. In another embodiment of the present invention, the glia cells consist of astrocyte, microglia, oligodendrocyte cells or a combination thereof. In an embodiment, co-cultured glia and neuron cells consist only of astrocytes and neurons. In an embodiment, co-cultured glia cells and neurons consists only of astrocytes, neurons and microglia. In an embodiment, co-cultured glia and neuron cells further comprises stem cells at no more than about 50%, 40%, 30%, 20% 10%, 5%, 2%, 1% or 0.1% of the cells. In an embodiment, the glia cells and neuron co-culture further comprises stem cells at about the same percentage as in brains of normal mice, rat, dog or human brains. In an embodiment, co-cultured glia and neuron cells are substantially free of stem cells or pluripotent stem cells. In an embodiment, co-cultured glia and neuron cells do not comprise an organoid. In an embodiment, the co-culture of the present invention is a 2 D culture. In an embodiment, co-cultured glia microglia and/or neuron cells comprising the present invention comprise any combination of cells that can exist in mammal brains and have been processed by trituration process and/or centrifuge separation process.

In an embodiment of the present invention, GNCM comprises a combination of neuron medium (NM) and glia culture medium (GCM). In another embodiment of the present invention, NM comprises Neurobasal™ and glucose. NM may further comprise B-27 supplement and L-glutamine. In an embodiment of the present invention, NM comprises about 0.5× to about 1.5× Neurobasal™ (2,250 mg/L glucose), about 1,000 mg/L to about 2,000 mg/L glucose, about 0.5× to about 1.5× B-27 supplement (serum free), and about 1 mM to about 3 mM L-glutamine. In another embodiment of the present invention, NM comprises about 1× Neurobasal™ (2,250 mg/L glucose), about 1,500 mg/L glucose, about 1× B-27 supplement (serum free), and about 2 mM L-glutamine.

In an embodiment of the present invention, GCM comprises Dulbecco's Modified Eagle's Medium (DMEM). GCM may further comprise one or more antibiotics and/or fetal bovine serum (FBS). In an embodiment of the present invention, GCM comprises about 0.5× to about 1.5× DMEM (without glutamine) (2,250 mg/L glucose), about 0.5 to about 1.5× penicillin-streptomycin, and about 5% to about 15% FBS. In another embodiment of the present invention, GCM comprises about 1× DMEM (without glutamine) (2,250 mg/L glucose), about 1× penicillin-streptomycin, and about 10% FBS. In an embodiment, the GCM comprises less than 7.5%, 5%, 4%, 3%. 2%, 1% by weight or is substantially free of FBS.

In an embodiment of the present invention the GCM optionally comprises one or more antibiotics comprises aminoglycosides, ansamycins, carbapenems, cephalosporins, glycopeptides, lincosamides, lipopeptides, macrolides, monobactams, nitrofurans, oxazolidinones, penicillins, polypeptides, quinolones, sulfonamides, tetracyclines, or others, or a combination thereof. In a preferred embodiment, the one or more antibiotics comprises penicillin and streptomycin. In another embodiment of the present invention, the GCM comprises less than about 10%, 7.5%, 5%, 2%, 1% or 0.1% by weight of antibiotics.

In an embodiment of the present invention, the glia-neuron culture medium (GNCM) of the present invention comprises a mixture of NM and GCM at a ratio of about 0.25:1 to about 4:1, about 0.5:1 to about 4:1, about 0.5:1 to about 3:1, about 1:1 to about 2:1. In another embodiment, GNCM comprises a mixture of NM and GCM at a ratio of about 1:1. In another embodiment, GNCM comprises a mixture of NM and GCM at a ratio of about 2:1.

In an embodiment of the present invention, the GNCM of the present invention further comprises FBS at about 0.5% to about 20% by weight. In another embodiment, the final concentration of FBS in GNCM is about 1.5% to about 15%. In a more preferred embodiment, the final concentration of FBS in GNCM is about 3% to about 10%. In another embodiment of the present invention, the system and method of the present invention is less than 3%, 2%, 1%, 0.5%, 0.1% or substantially free of FBS.

Other supplements that may be added include selenium, biotin, insulin, sodium pyruvate, hydrocortisone, BSA(bovine serum albumin), triiodothyronine (T3), apo-transferrin, n-acetylcysteine (NAC), putrescine etc. . . .

In another embodiment of the present invention comprises a modified glia-neuron culture medium (mGNCM) which uses modified neuron medium (mNM) rather than NM described above so that mGNCM is a combination of mNM and GCM. In an embodiment, the mNM comprises about 0.50× to about 1.5×(Glucose:2250 mg/L), about 1000 to about 2000 mg/L glucose, about 25× to about 75× B-27 plus supplement and about 50× to about 150× GlutaMAX supplement. In an embodiment, the mNM comprises about 1.0×(Glucose:2250 mg/L), about 1500 mg/L glucose, about 50× B-27 plus supplement and about 100× GlutaMAX supplement.

In an embodiment of the present invention, the modified glia-neuron culture medium (mGNCM) of the present invention comprises a mixture of mNM and GCM at a ratio of about 0.25:1 to about 4:1, about 0.5:1 to about 4:1, about 0.5:1 to about 3:1, about 1:1 to about 2:1. In another embodiment, mGNCM comprises a mixture of mNM and GCM at a ratio of about 1:1. In another embodiment, mGNCM comprises a mixture of mNM and GCM at a ratio of about 2:1.

Other supplements that may be added include selenium, biotin, insulin, sodium pyruvate, hydrocortisone, BSA(bovine serum albumin), triiodothyronine (T3), apo-transferrin, n-acetylcysteine (NAC), putrescine etc. . . .

As mentioned above, both GNCM and mGNCM of the present invention may be applied to various animal and/or human models that provides cell morphology, cell reaction and/or interaction between the cells that occur in vivo. Cells cultured within any embodiment of the GNCM and mGNCM described above can be maintained for up to at least 21 days, at least 30 days, at least 40 days, at least 45 days and still retain said morphology, cell reaction and cell interaction useful for conducting animal and/or human models such as in research, drug screening, tests or clinical trials in various study areas such as oxygen-glucose deprivation, ischemic stroke, inflammation, cell death, gliosis, ischemic stroke, encephalitis, and neurodegenerative disease, such as Parkinson disease and Alzheimer's disease, Huntington's disease, multiple sclerosis, amyotrophic lateral sclerosis, vascular dementia etc. . . . .

In another embodiment of the present invention, the cell culture medium used for co-culturing glia cells and neurons comprises only GCM. In another less preferred embodiment, the cell culture medium used for co-culturing glia cells and neurons comprises only NM or mNM.

The present invention also provides a method for modeling glia and neuron cell morphology, cell reaction and cell interaction using any of the embodiments of glia cells described above as well as any of the embodiment so the GNCM and mGNCM described above in research, drug screening, testing and clinical trials for studies related to oxygen-glucose deprivation, ischemic stroke, inflammation, cell death, gliosis, ischemic stroke, encephalitis, and neurodegenerative disease, such as Parkinson disease and Alzheimer's disease, Huntington's disease, multiple sclerosis, amyotrophic lateral sclerosis, vascular dementia etc. . . . The glia cells and neurons may be obtained from mammals typically used for research, drug screening, tests and clinical trials such as mice, rats, dogs, non-human primates, sheep, cows or human beings. In an embodiment of the method present invention for animal modeling, the glia cells and neurons may be obtained from the neonatal brain primary cultures of rats or mice, which is useful for conducting the functional study of animal brains. Notably, cells cultured with the method of the present invention for fewer days such as about 5 days, about 7 days or about 10 days can be used to simulate in vivo conditions of an immature brain, while cells cultured for more days such as about 20 days, 25 days, 30 days, 40 days, 45 days or more can be used to simulate in vivo conditions of a brain at various stages of maturity.

The present invention also provides a method for preparing any embodiments of the GNCM and mGNCM glia cells and neuron co-culture described comprising the steps of triturating the cerebral cortex in any embodiment of GCM described, stationing for 1 minute at room temperature to obtain a supernatant, centrifuging the supernatant at 1,500 rpm to obtain the cell pellet, resuspending the pellet in the GCM, seeding the solution in a 24-well plate coated with poly-L-lysine (0.1 mg/mL) for 1 hour, then replacing the GCM with any embodiment of the GNCM or mGNCM described above. Cells co-cultured with the method of the present invention can be maintained for at least up to 21 days, at least 30 days, at least 40 days, at least 45 days and still maintain cell morphology, cell reaction and cell interaction similar to in vivo conditions useful for conducting animal and/or human models.

In an embodiment of the method of preparation of the present invention, the glia cells and neurons are obtained from the brains of mammals usually used in research, drug screenings, tests and clinical trials such as rats, mice, dogs, non-human primates, sheep, cows or human beings. In another embodiment of the present invention, the glia cells and neurons are obtained from neonatal brain primary cultures of rats or mice. In yet another embodiment of the present invention, the co-cultured glia cells and neurons comprise astrocytes and neurons.

A method of preparing the GNCM of the present invention comprises combining a Composition A, a Composition B, and a Composition C. In an embodiment, Composition A comprises about Neurobasal™ medium (glucose: 4,500 mg/L) and 1,500 mg/L glucose. Composition A is preferably stored at 4° C. In an embodiment, the Composition B comprises high glucose DMEM (glucose: 4,500 mg/L) that does not contain glutamine. Composition B is preferably stored at 4° C. In an embodiment, Composition C comprises B27™ Supplement (serum free) with optional FBS and penicillin-streptomycin. Composition A, B, and C are combined in various ratios to achieve any of the embodiments described above with about 5%, 10% CO₂ and warmed up to about body temperature then to which any embodiment of the glia cells and neurons described are added.

A method for preparing the mGNCM of the present invention comprises combining a Composition A, a Composition B and a Composition C with any embodiment of the glia cells and neurons described. In an embodiment, Composition A comprises about Neurobasal™ medium (glucose: 4,500 mg/L), 1,500 mg/L glucose and 100× Glutamax™ supplement. Composition A is preferably stored at 4° C. In an embodiment, the Composition B comprises high glucose DMEM (glucose: 4,500 mg/L) that does not contain glutamine. Composition B is preferably stored at 4° C. In an embodiment, Composition C comprises 50×B-27™ Plus Supplement (serum free) with optional FBS and penicillin-streptomycin. Composition C is preferably stored at −20° C. Composition A, B, and C are combined in various ratios to achieve any of the embodiments described above with about 5%, 10% CO₂ and warmed up to about body temperature then to which any embodiment of the glia cells and neurons described are added.

Other supplements that may be added include selenium, biotin, insulin, sodium pyruvate, hydrocortisone, BSA(bovine serum albumin), triiodothyronine (T3), apo-transferrin, n-acetylcysteine (NAC), putrescine etc. . . .

### Examples

### Example 1: Glia-Neuron Culture Medium (GNCM) Glia Cells and Neurons Co-Culture

TABLE 1

| | | | Company |
|---|---|---|---|
| | | Final | (catalog |
| | Components | Concentration | number) |
| Neuron | Neurobasal ™ | 0.5x | Gibco |
| medium (NM) | (1x) liquid | (glucose: | (21103049) |
| | (Glucose: 4,500 mg/L) | 2,250 mg/L) | |
| | Glucose | 750 mg/L | Sigma |
| | (1,500 mg/L) | | (G8270) |
| | 1x B-27 supplement, | 0.5x | Gibco |
| | serum free | | (17504044) |
| | 2 mM L-glutamine | 1 mM | Gibco |
| | | | (A2916801) |
| Glia culture | DMEM, | 0.5x | Gibco |
| medium (GCM) | high glucose | (Glucose: | (11960) |
| | (4,500 mg/L), | 2,250 mg/L) | |
| | no glutamine | | |
| | 1x | 0.5x | Corning |
| | Penicillin- | | (30-002-CI) |
| | streptomycin | | |
| | (P/S) | | |
| | 10% FBS | 5% | Hyclone |

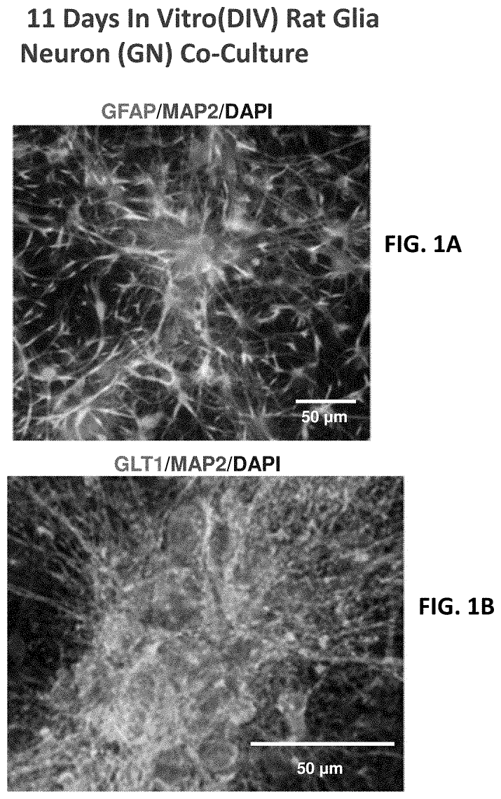

An embodiment of the GNCM composition of the present invention prepared by combining the components of Table 1 along with glia cells and neurons obtained from rat neonatal brain primary culture. The resulting cells maintained for about 12 to about 14 days. At 11 days in vitro, the mixed culture was dyed for the observation of cell morphology. FIG. 1A shows astrocyte cytoskeleton marked with GFAP and colored in green, neuron dendrites marked with MAP2 and colored in red, as well as cell nuclei marked with DAPI and colored in blue. FIG. 1A indicates (i) neuron forms cluster in the GN culture system similar to morphology of the brain nucleus that exist in the brain in vivo. In addition, astrocyte surrounding the neuronal cluster in FIG. 1A also indicates astrocyte and neuron interaction in the GNCM culture of the present invention similar to the structure in vivo. FIG. 1B shows astrocyte transporters marked with GLT1 and colored in green, neuron dendrites marked by MAP2 and colored in red, as well as cell nuclei marked with DAPI and colored in blue. FIG. 1B illustrates that astrocyte processes tightly enwrapping neuronal dendrites closely resembling perineuronal astrocyte formation in vivo.

### Example 2: Modified Glia-Neuron Culture Medium (mGNCM) Glia Cells and Neurons Co-Culture

TABLE 2

| | | | Company |
|---|---|---|---|
| | | Final | (catalog |
| | Components | Concentration | number) |
| Neuron medium | Neurobasal ™ | 0.5x | Gibco |
| (NM) without | medium | (Glucose: | (21103049) |
| supplement | (1X) liquid | 2,250 mg/L) | |
| | (Glucose: 4,500 mg/L) | | |
| | Glucose | 750 mg/L | Sigma |
| | (1,500 mg/L) | | (G8270) |
| Glia culture | DMEM, | 0.5x | Gibco |
| medium (GCM) | High glucose | (Glucose: | (11960) |
| | (4,500 mg/L), | 2,250 mg/L) | |
| | No glutamine | | |
| | 1s Penicillin- | 0.5x | Corning |
| | streptomycin | | (30-002-CI) |
| | (P/S) | | |
| | 10% FBS | 5% | Hyclone |
| Glia-Neuron | 50x B-27 | 1x | Gibco |
| culture | Plus supplement | | (A3582801) |
| supplement | 100x Glutamax ™ | 1x | Gibco |
| | supplement | | (35050061) |

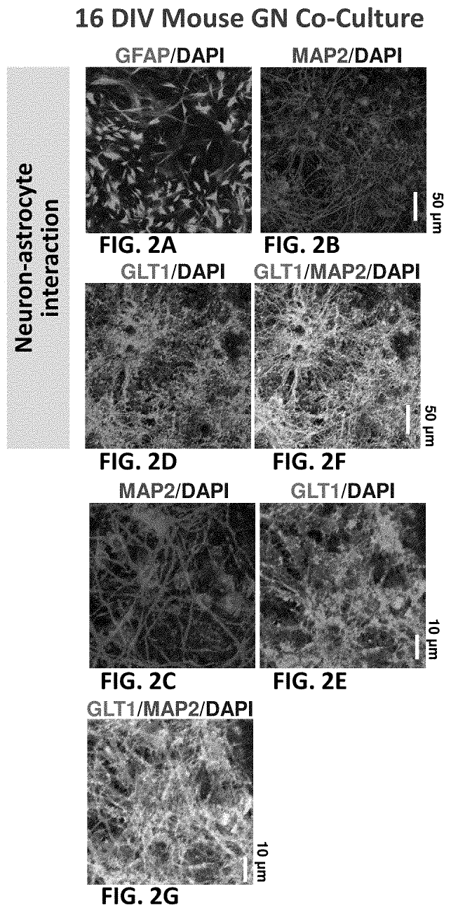

An embodiment of the mGNCM composition of the present invention prepared by combining the components of Table 2 along with glia cells and neurons obtained from mouse neonatal brain primary culture. The resulting cells maintained for about 16 to about 21 days. At 16 days in vitro, the mixed culture of mouse brain cells was dyed for the observation of cell morphology and neuron-glia interaction. FIG. 2A shows astrocyte cytoskeleton marked with GFAP and colored in green, and cell nuclei marked with DAPI and colored in blue. FIGS. 2B and 2C show neuron dendrites marked with MAP2 and colored in red, as well as cell nuclei marked with DAPI and colored in blue, in different scales. FIGS. 2D and 2E show astrocyte transporters marked with GLT1 and colored in green, as well as cell nuclei marked with DAPI and colored in blue, in different scales. FIGS. 2F and 2G show astrocyte transporters marked with GLT1 and colored in green, neuron dendrites marked with MAP2 and colored in red, as well as cell nuclei marked with DAPI and colored in blue, in different scales. As can be seen, the astrocytes exhibit long, thin morphology similar to that of astrocytes in vivo. Moreover, astrocyte transporters marked with GLT1 indicate the formation of thin astrocytic processes which enwrap neurons, a structure that exists in vivo as well.

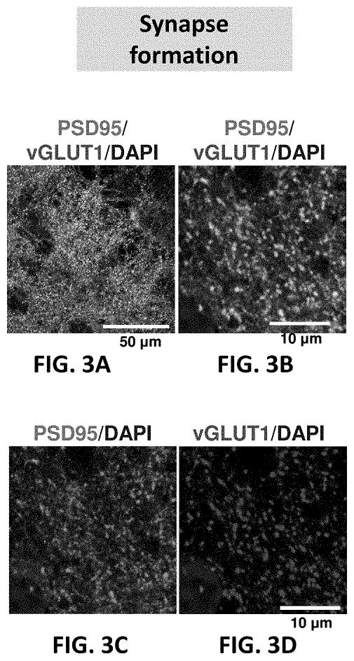

The fluorescent staining images of FIG. 3 are indications of synapse formation, wherein presynaptic axon terminals are marked with PSD95 and colored in green, neuron dendrite pre-synapses are marked with vGLUT1 and colored in red, cell nuclei are marked with DAPI and colored in blue, and the overlapping regions of post-synapses and pre-synapses are colored in yellow. The closeness between pre-synapses and post-synapses indicate possible sites of synapse formation as exists in vivo.

### Ischemia Modeling

### Example 3: Oxygen-Glucose Deprivation Ischemic Stroke In Vitro Model

Glia cells and neurons obtained from mouse primary neonatal brain cells cultivated using the mGNCM composition of the present invention were cultivated for 14 days. At 14 DIV, the mixed culture was subjected to oxygen-glucose deprivation (OGD) where the GN cells were changed to glucose-free EBSS and incubated in 1% 02 for 1 hour, followed by recovery in normal 21% oxygen and maintain medium (mGNCM) for 23 hours. Fluorescent staining images of the cultures at normoxia and under OGD conditions are shown in FIGS. 4A and 4B, and FIGS. 4C and 4D, respectively. FIGS. 4A and 4C show neuron dendrites marked with MAP2 and colored in red, as well as cell nuclei marked with DAPI and colored in blue. FIGS. 4B and 4D show microglia marked with Iba1 and colored in green, as well as cell nuclei marked with DAPI and colored in blue. As can be seen in the figures, the number of neurons decreased following OGD treatment which is also seen in neurons in vivo following OGD treatment immediately below. In addition, microglia, cells responsible for immune responses in the brain, have transformed from thinner resting microglia to more hypertrophic activated microglia which is also seen in neurons in vivo following OGD treatment immediately below. From this example of the present invention, an embodiment of utilizing the glia-neuron co-culture system of the present invention as an in vitro model in simulation of brain cell physiology.

A mouse in vivo ischemic stroke model, transient hypoxia-ischemia (tHI), is used to demonstrate similar effects of hypoxic ischemia in the hippocampus brain region. The tHI mice were anesthetized by 1% isoflurane during the surgical procedure of right common carotid artery (rCCA) isolation and ligation. Sham-operated pups were subjected to the same procedures of anesthesia, surgical incision, and right CCA isolation. The mice subjected to 7.5% oxygen and 92.5% nitrogen for 25 min for hypoxic insult. At the end of hypoxia, the mice were recovered in room air with the knots on the right CCA for 3 days reperfusion. FIGS. 4E and 4F shows immunofluorescent images of the hippocampal CA1 and CA3 subregions under both sham and tHI conditions, with astrocytes marked with GFAP and colored in green, and microglia marked with Iba1 and colored in red. As can be seen in FIGS. 4E and 4F, in both the CA1 and CA3, tHI insult induces astrocyte and microglia hypertrophy.

Similarly, a rat trial of chronic cerebral hypoperfusion (CCH) using hypoxia-sensitized unilateral common carotid artery occlusion is performed to demonstrate the similarities of in vivo responses and those of brain cells cultured in the co-culture system of the present invention. Hypoxia-sensitized Common Carotid Artery Occlusion (HUCCAO) was established by permanent occluded of right common carotid artery and sham-operated animals served as control. After artery ligation, the anesthetized air was switch into hypoxia gas cylinder (7.5% O2/92.5% N2) for 30 minutes under anesthesia (with 1.5% isoflurane). Rats were sacrificed at PID 7 for immunohistochemical staining. FIGS. 4G and 4H show immunofluorescent images of rat cingulate cortex, with Iba1 used as a marker for microglia, colored in green. As can be seen in the difference between the sham and CCH groups, CCH induces microglia proliferation and hypertrophy in rat cingulate cortex, similar to results of the in vitro model of the present invention.

### Example 4

In another embodiment, the brains of adult mice were used to study the effect of aryl hydrocarbon receptor modulation on stroke-induced astrogliosis and neurogenesis. The study is published in Chen W, Chang L H, Huang S S, Huang Y J, Chih C L, Kuo H C, Lee Y H and Lee I H Aryl hydrocarbon receptor modulates stroke-induced astrogliosis and neurogenesis in the adult mouse brain. Journal of Neuroinflammation. 2019 16:187 and is hereby incorporated in its entirety.

The aryl hydrocarbon receptor (AHR) is a ligand-dependent transcription factor activated by environmental agonists and dietary tryptophan metabolites for immune response and cell cycle regulation. Emerging evidence suggests that AHR activation after acute stroke may play a role in brain ischemic injury. To examine whether AHR activation alters poststroke astrogliosis and neurogenesis, conditional knockout of AHR from nestin-expressing neural stem/progenitor cells (AHRcKO) and wild-type (WT) mice in the permanent middle cerebral artery occlusion (MCAO) model, an ischemic stroke model, was used. WT mice were treated with either vehicle or the AHR antagonist 6,2′,4′-trimethoxyflavone (TMF, 5 mg/kg/day) intraperitoneally. The animals were examined at 2 and 7 days after MCAO. At 48 h after MCAO, microglial and astroglial activation was observed.

At 48 h after MCAO, immunohistochemical analyses revealed that the AHR protein was robustly expressed in ionized calcium-binding adapter molecule 1 (Iba1)-positive microgliosis and glial fibrillary acidic protein (GFAP)-positive astrogliosis predominantly at the peri-infarct cortex of the vehicle-treated WT, AHR^{fix/fix} mice compared to the normal WT and the normal AHRcKO, respectively. Moreover, the TMF-treated WT and AHRcKO mice showed markedly decreased GFAP-positive astrogliosis and Iba1-positive microgliosis at the peri-infarct cortex after MCAO. Notably, AHRcKO showed spared and reduced AHR immunoreativities in Iba1-positive microglia that represented the effector cell population in response to acute ischemic stroke and neuroglial deletion of AHR. These findings suggest that inhibition and the nestin⁺ cell-specific knockout of AHR reduced poststroke astrogliosis and microgliosis, indicating the reduced innate immune responses after stroke.

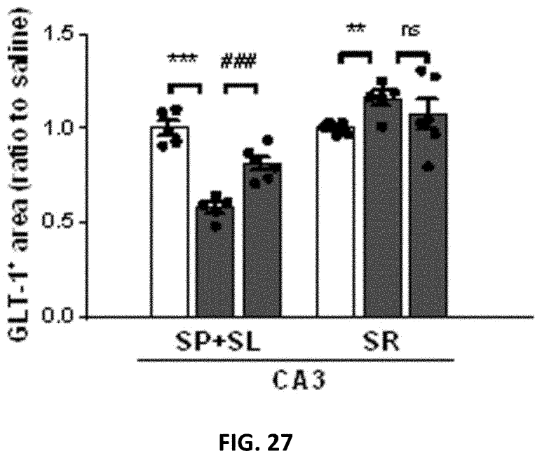

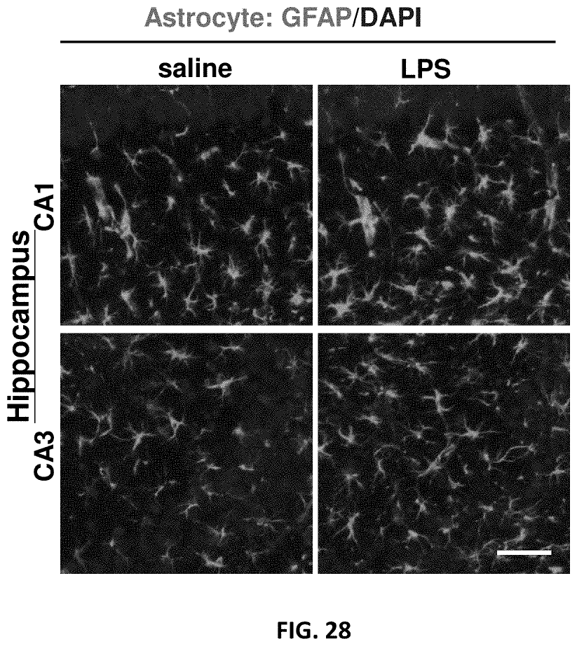

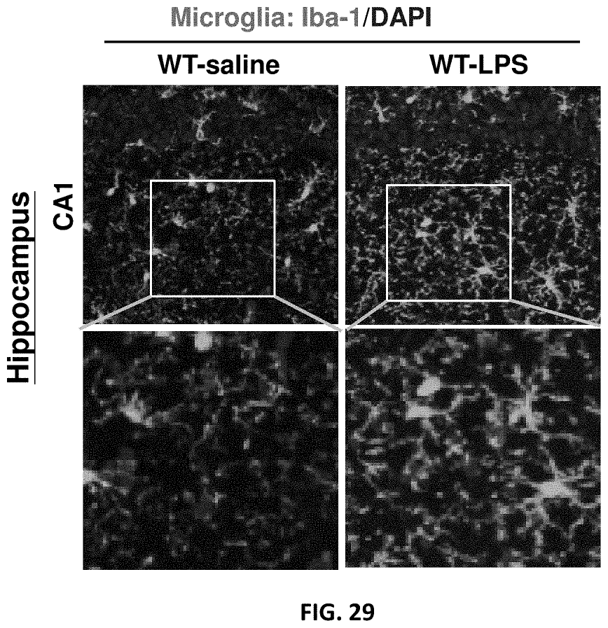

FIGS. 41 and 4J show representative immunohistochemical staining for neuroinflammation at the peri-infarct cortex (bregma=+0.86 mm) of normal WT (n=6/each group), poststroke WT-Vehicle treated, WT-TMF treated, normal AHRcKO, poststroke AHR^{fix/fix}, and AHRcKO mice. The inset in low magnification is shown with a high magnification view in the next row(s). At high magnification, note the increase in AHR immunoreactivity colocalized within Iba1-positive microglia and GFAP-positive astrocytes (red arrows) in the WT-Vehicle-treated and AHR^{fix/fix} mice after MCAO, which was markedly decreased in the WT-TMF-treated and ARHcKO mice (whose AHR deletion was spared in microglia), respectively. Furthermore, increased Iba1-positive microgliosis and GFAP-positive astrogliosis in the WT-Vehicle and AHR^{fix/fix} mice after MCAO were significantly decreased in the WT-TMF-treated and AHRcKO mice (whose AHR was deleted in astroglia), respectively. Intracellular AHR immunoreactivity was predominantly in the nucleus, and to a lesser extent in the cytoplasm. The scale bars represent 60 μm at low magnification and 8 μm at high magnification.

FIGS. 4K, 4L and 4M show quantifications of the relative immunereactive stained with AHR, Iba-1, and GFAP, respectively. As shown in FIGS. 41-M, microglial and astroglial activation are results of MCAO in mice, similar to the results of FIG. 4A-D.

### Inflammation Modeling

### Example 5 LPS Neuroinflammation

TABLE 3

| | Neuron | Fetal | | 100x |
|---|---|---|---|---|
| | medium:Glia | bovine | 50x B-27 ™ Plus | Glutamax ™ |
| | culture medium | serum | supplement | Supplement |
| 1:1 group | 1:1 | 5% | 1/50 (1x) | 1/100 (1x) |
| 2:1 group | 2:1 | 3.3% | 1/50 (1x) | 1/100 (1x) |
| GCM only | Glia culture | 10% | — | — |
| group | medium only | | | |

The efficacies of simulating in vivo cell morphology, cell interactions and responses to stimuli in vitro using mGNCM of different ratios of modified neuron medium (mNM) and glia culture medium (GCM) were compared, 1:1:, 2:1 and GCM only groups, and the GCM only group comprising only GCM and 10% FBS as shown in Table 3 above.

The effects of neuroinflammation simulation of rat cerebral cortex cells grown in vitro using the three embodiments of mGNCM of Table 3 were examined. Cells obtained from rat cerebral cortex were cultured in vitro for 13 days using the three culture media above, then treated with 1 μg/mL of lipopolysaccharides (LPS) for 24 hours on day 13 to trigger inflammation. Following LPS treatment, immunostaining was performed to observe cell population, morphology, and response to inflammatory stimulus.

The immunostaining images of FIG. 5 show neuronal population and response to LPS stimulation, wherein CTL represents control groups (not subjected to LPS treatment) and LPS represents groups treated with LPS to induce neuroinflammation. The images show astrocyte cytoskeleton marked with GFAP and colored in red, neuron dendrites marked with MAP2 and colored in green, as well as cell nuclei marked with DAPI and colored in blue. FIGS. 5A1-5A3 and 5B1-5B3 show cells cultured with GCM only, FIGS. 5C1-5C3 and 5D1-5D3 show cells cultured with 1:1 mNM and GCM, while FIGS. 5E1-5E3 and 5F1-5F3 show cells cultured with 2:1 mNM and GCM. The GCM only group comprises fewer neurons, and the cells are smaller and unhealthier in both CTL and LPS treatment groups. Of the 1:1 group, neurons of the CTL group exhibit the morphology of healthy neurons, while signs of neuron damage and some cell debris in staining can be found in the LPS treatment group, indicating neuroinflammation. Of the 2:1 group, neurons of the CTL group exhibit the morphology of healthy neurons and are surrounded by more astrocytes while being less clustered. Though the number of neurons in the LPS treatment group does not seem to have decreased, the images show possible fiber sprouting, which may have been caused by inflammation.

FIG. 6 shows the quantified results of cell growth of FIG. 5. FIG. 6A graphs the percentages of MAP2 positive cells in the total population, FIG. 6B graphs the percentages of GFAP positive cells in the total population, while FIG. 6C graphs the astrocyte/neuron ratios of each group. As shown in FIG. 6A, while LPS stimulation induces neuronal loss in the 1:1 group, the GCM only and 2:1 groups are not affected in this way. As such, 1:1 mGNCM culture conditions are ideal for the study of inflammation-induced neuro toxicity and neurodegeneration.

The astrocyte/neuron ratio in the brain vary widely as known in the art and disclosed in Herculano-Houzel S. The Glia/Neuron Ratio: How it Varies Uniformly Across Brain Structures and Species and What that Means for Brain Physiology and Evolution. DOI: 10.1002/glia/22683 and is hereby incorporated in its entirety. As can be seen in FIG. 6C, astrocyte/neuron ratios correlate reversely to the proportion of NM in the culture medium. Therefore, the ratio of NM and GCM as well as ratio of mNM and GCM may be adjusted to simulate astrocyte/neuron ratios of different brain regions. Notably, the astrocyte/neuron ratios of the 1:1 group and the GCM only group result in about 5:1 and about 10:1 astrocyte/neuron ratios, respectively, which lie within normal ranges found in the brains of rats.

FIG. 7 shows the morphological differences of astrocytes of different groups, with astrocyte cytoskeleton marked with GFAP and colored in red, as well as cell nuclei marked with DAPI and colored in blue. Astrocyte hypertrophy induced by LPS stimulation can be found in both the 1:1 group and 2:1 group, as shown in FIGS. 7D and 7F, but the same effect is not observed in the GCM only group, as shown in FIG. 7B. Moreover, inflammation-induced astrogliosis is more pronounced in the 1:1 and 2:1 groups, making these groups good approximations of neuroinflammation of brain cells grown in vivo. Notably, though the GCM only group comprises more glia cells, its response to inflammatory stimulus is inferior to that of the 1:1 and 2:1 groups, as shown in FIGS. 7A and 7B.

FIG. 8 compares the density and morphology of microglia in the hippocampus of mice and in cell culture media of the present invention, wherein neuron dendrites are marked with MAP2 and colored in green, microglia are marked with Iba1 and colored in red, and cell nuclei are marked with DAPI and colored in blue. As shown in FIG. 8E, the cells of the 1:1 group exhibit thin processes like those of the hippocampus saline group, shown in FIG. 8A. Moreover, as shown in FIG. 8F, the reaction to LPS of the 1:1 group is more similar to that of the hippocampus group, shown in FIG. 8B. Thus, cells cultured with the 1:1 mNM and GCM medium are well suited to simulation of cells cultured in vivo, as well as to simulation of morphology and behavior of microglia grown in vivo. FIG. 8G shows that microglia of the 2:1 group exhibit unhealthier morphology as compared with those of the hippocampus and 1:1 groups. Though the reaction of the 2:1 group to LPS stimulus, shown in FIG. 8H, appears to be more consistent, it may be a result of its neurons' unhealthier baseline condition.

FIG. 9 quantifies the results of fluorescent imaging of FIG. 8, with FIG. 9A graphing the percentage of Iba1 positive cells in the total count of DAPI stains, namely the percentage of microglia in total cells, and FIG. 9B showing the Iba1 positive area per cell, the size of the microglia. As can be seen, the 1:1 group is superior on both counts compared to the GCM only and 2:1 groups.

Our studies demonstrate that cells cultured in mGNCM (the 1:1 NM and GCM group) are more suitable for the in vitro culture of cerebral cortex to simulate the in vivo environment and physiological reactions of the brain, while cells cultured in the 2:1 NM and GCM medium are useful in simulation of the interaction between neurons and astrocytes.

### Example 6 LPS Neuroinflammation

TABLE 4

| Neuron | Fetal | | |
|---|---|---|---|
| medium:Glia | bovine | B-27 ™ Plus | GlutaMAX ™ |
| culture medium | serum (%) | supplement | Supplement |
| GCM only | 5 | 1/50 (1x) | 1/100 (1x) |
| 0.5:1 | 5 | 1/50 (1x) | 1/100 (1x) |
| 1:1 | 5 | 1/50 (1x) | 1/100 (1x) |
| (mGNCM) | | | |
| 2:1 | 5 | 1/50 (1x) | 1/100 (1x) |
| 4:1 | 5 | 1/50 (1x) | 1/100 (1x) |
| mNM only | 5 | 1/50 (1x) | 1/100 (1x) |

To further examine the effects of various embodiments of mGNCM with different ratios of mNM and GCM of the present invention on cell type population and morphology of rat glia cells and neurons co-culture, six cell culture media were prepared to the specifications of Table 4.

On postnatal day 0 (P0), cells extracted from rat cerebral cortex were inoculated in each of the cell culture media of Table 4. Following 14 days of in vitro cultivation, immunostaining was performed on the control groups (CTL) to aid in the observation of cell type ratios, specifically of neurons, astrocytes, and microglia, and oligodendrocytes as well as cell morphology. Cultures of the LPS groups (LPS) were subjected to inflammatory stimulation via 1 μg/mL LPS at 13 days in vitro. After another 24 hours, the LPS groups were subjected to immunostaining as well, for the observation of cell morphology and cell count.

FIGS. 10 and 11 show immunostaining results of both control and LPS groups of cells cultivated in GCM only medium, mNM:GCM 0.5:1 medium,m NM:GCM 1:1 medium, mNM:GCM 2:1 medium, mNM:GCM 4:1 medium, and mNM only medium. The images show astrocyte cytoskeleton marked with GFAP and colored in red, neuron dendrites marked with MAP2 and colored in green, as well as cell nuclei marked with DAPI and colored in blue. As illustrated in the figures, LPS stimulation induces astrocyte hypertrophy and process arborization. In addition, the addition of NM promotes the formation of thinner astrocyte processes. As seen in FIGS. 10A-10F, co-cultures grown in GCM only medium produce fewer neurons, and that the neuronal processes are unnaturally fat and short. As astrocyte morphology is closely linked to that of neurons, the astrocytes of the GCM only group exhibit abnormal morphology. Some reaction to LPS may be seen, likely the result of glia response. Co-cultures grown in mNM:GCM 0.5:1 medium, shown in FIGS. 10G-10L, exhibit improved cell morphology over the GCM only group, though neuronal processes are still shorter than their in vivo state. LPS treatment induces neuron outgrowth in this group, and unhealthy neurite stress fibers similar to the morphology of more aged cell cultures are observed. As FIGS. 10M-10R show, neuron morphology is more relaxed and natural when the co-culture is cultivated in mNM:GCM 1:1 medium. The co-cultures of the GCM only group, the 0.5:1 group, and the 1:1 group all react to LPS, though neurons are more impacted, and some induced neuroplasticity can be observed. FIGS. 11A-11F show the immunostaining results of the mNM:GCM 2:1 group. Some neuron damage can be observed following LPS treatment. FIGS. 11G, 11I, and 11K show that neurons cultured in mNM:GCM 4:1 medium without LPS treatment exhibit unhealthy morphology. Some response to LPS treatment can be seen in FIGS. 11H, 11J, and 11L. In the mNM only group, shown in FIGS. 11M-11R, neurons form clusters and fewer processes, unlike their natural, in vivo, state.

FIG. 12 shows the percentage of MAP2-circled cells in total DAPI, meaning the percentage of neurons in total cells, for each of the groups of Table 4. Interestingly, though the presence of mNM increases neuron percentage, higher ratios of mNM in the co-culture media don't necessarily produce larger neuron populations, likely because neurons do not proliferate, and that their cell count depends on initial seeding. As can be seen, LPS treatment reduced the percentage of neurons in the co-culture systems of all groups except for the GCM only group. This could be a combined result of cell death due to inflammatory neurotoxicity and glia proliferation. In addition, significant neuronal loss following LPS treatment may be observed in the 2:1 mGNCM group, indicating that the 2:1 medium is useful in simulating LPS-induced neurotoxicity and neurodegeneration that can be observed in vivo. Therefore, the mNM:GCM 2:1 co-culture system may be used as a screening platform for neurodegeneration caused by infectious diseases.

FIG. 13 shows the percentage of GFAP-circled cells in total DAPI, meaning the percentage of astrocytes in total cells, of each of the groups of Table 4. As can be seen, LPS treatment does not cause significant proliferation or death of astrocytes, indicating that the decrease in neuron percentage observed in FIG. 12 is mainly a result of neuronal death.

FIG. 14 shows the ratio of astrocytes to neurons of each of the groups of Table 4. As can be seen, the astrocyte to neuron ratios range from about 2:1 to about 6:1, which fall within the range that can be found in vivo. Notably, in the 2:1 group, there is a significant difference of astrocyte to neuron ratio between the control group and LPS group, indicating significant neurodegeneration, as stated above.

FIGS. 15 and 16 show immunostaining results of both control and LPS groups of cells cultivated in GCM only medium, mNM:GCM 0.5:1 medium, mNM:GCM 1:1 medium, mNM:GCM 2:1 medium, mNM:GCM 4:1 medium, and mNM only medium. The images show microglia marked with Iba1 and colored in red, neuron dendrites marked with MAP2 and colored in green, as well as cell nuclei marked with DAPI and colored in blue. As can be seen, LPS stimulation induces microglial hypertrophy (microgliosis) in all groups except for the GCM only group, in which the microglia in resting state, namely those of the control group, are already overly active. Also notable is the fact that resting microglia of the NM only group are a round, unhealthy shape, different from that of their natural, in vivo morphology. FIGS. 16A-16F show that in the mNM:GCM 2:1 co-culture system, LPS induces an increase of microglia, indicating microglial proliferation or recruitment. FIGS. 16G-16L show that microglia of the control group are more active, indicating that the neurons are sickly, and that the reaction to LPS treatment is diminished as compared to the 0.5:1, 1:1, and 2:1 groups.

FIG. 17 shows the percentage of Iba1 positive area per cell, meaning the size of microglia per culture area, of each group of Table 4. As noted above, microglia morphology of the 1:1, 2:1, and 4:1 groups show hypertrophy under LPS stimulation, the results of which are confirmed in FIG. 17 by significant differences of Iba1 positive areas per cell between the control groups and LPS groups.

FIG. 18A shows the percentage of Iba1 positive cells over in total DAPI, meaning the percentage of microglia in total cells, of each group of Table 4. As can be seen, there is a significant difference in microglia population between the control and LPS groups of the 1:1 group, indicating that LPS induces significant microglia proliferation only in mNM:GCM 1:1 medium. In addition, microglial population in all medium conditions are in the range of 7-15%, similar to the population in the brain. The microglial population in the total population of brain cells and show similar adult rodent brain accounts for 5 to 12% of the total number of cells1. In the human brain, microglia account for 0.5 to 16.6% of regional variability to that reported in rodents. Local distribution of microglia in the normal adult human central nervous system differs by up to one order of magnitude. Mittelbronn M, Dietz K, Schluesener H J, Meyermann R Acta Neuropathol. 2001 Mar; 101(3):249-55. Turnover of resident microglia in the normal adult mouse brain. Lawson L J, Perry V H, Gordon S Neuroscience. 1992; 48(2):405-15.

FIG. 18B shows the morphological plasticity of oligodendrocyte in response to 1 μg/ml LPS treatment in GN mix culture. FIG. 18C is a quantification result of FIG. 18B.

The two figures indicate that though LPS does not affect oligodendrocyte (OL) cell density among different groups of GN mix cultures, the oligodendrocyte process shrinkage induced by LPS can be observed in both mNM:GCM 0.5:1 and 1:1 media. These results indicate that the media can be applied to study and inflammation-induced oligodendrocyte damage in demyelinating diseases.

### Example 7

An embodiment of the present invention was used in the study of soluble epoxide hydrolase (sEH) inhibition and its regulation of glutamate excitotoxicity in a stroke-like model. The study is described in Kuo Y M, Hsu P C, Hung C C, Hu Y Y, Huang Y J, Gan Y L, Lin C H, Shie F S, Chang W K, Kao O S, Tsou M Y, Lee Y H. Soluble Epoxide Hydrolase Inhibition Attenuates Excitotoxicity Involving 14,15-Epoxyeicosatrienoic Acid-Mediated Astrocytic Survival and Plasticity to Preserve Glutamate Homeostasis Molecular Neurobiology Jul. 1, 2019. and is hereby incorporated in its entirety.

Astrocytes play pivotal roles in regulating glutamate homeostasis at tripartite synapses. Inhibition of soluble epoxide hydrolase (sEHi) provides neuroprotection by blocking the degradation of 14,15-epoxyeicosatrienoic acid (14,15-EET), a lipid mediator whose synthesis can be activated downstream from group 1 metabotropic glutamate receptor (mGluR) signaling in astrocytes. In a study, glia-neuron (GN) co-cultures using an embodiment of GNCM of the present invention, neuron-enriched (NE) cultures, purified astroctye cultures, as well as excitotoxicity brain injury murine models were used to investigate the role and mechanism of 14,15-EET and sEH inhibition in excitotoxic brain injury. 14,15-EET and sEH inhibition were found to mediate astroglial protection against excitotoxicity, an effect critical to their glia-dependent neuroprotective effect, as they attenuated excitotoxicity-induced disruption of perineuronal astrocyte processes and GLT-1-mediated glutamate homeostasis.

Primary cortical NE cultures were prepared from embryonic day 17 SD fetal rats. The cultures were maintained in Neurobasal™ medium, and 9-10 days in vitro (DIV) culture was used for the study. Primary astrocyte cultures were prepared from SD rat pups at P1-2, with the purified astrocytes grown in 50% DMEM/50% Neurobasal™ medium containing heparin-binding EGF-like growth factor (HBEGF) or GNCM wherein GCM and NM are at a ratio of 1:1. Primary glia-neuron mix cultures were prepared from P0 rat brains. The harvested cerebral cortex was mechanically triturated in glia culture medium (GCM), comprising DMEM supplemented with 10% FBS. The mixture was stationed at room temperature for 1 minute, and the supernatant centrifuged at 1,500 rpm to obtain the cell pellet. The pellet was resuspended in GCM and seeded onto 24-well plates coated with poly-L-lysine (0.1 mg/mL). The culture medium was replaced with an embodiment of the GNCM medium of the present invention comprising a 1:1 mixture of GCM and B-27-containing Neurobasal™ 1 hour after the initial seeding.

Oligodendrocytes obtained from mouse primary neonatal brain cells cultivated using the GN culture compositions provided in table 4 were treated with 5 μM NMDA at 14 DIV for 24 hour.

The day of cell plating was considered as 0 DIV, and 9-10 DIV cultures were used for the experiments. The NE culture contained approximately 30% astrocytes and 70% neurons, and the GN culture contained approximately 85% astrocytes and 15% neurons, as characterized by immunolabeling with microtubule-associated protein 2 (MAP2) shown in red, and glial fibrillary acidic protein (GFAP) shown in green for neurons and astrocytes, respectively, as well as DAPI immunostaining for cell nuclei, shown in blue, as demonstrated in FIG. 19. The abundances of neurons versus astrocytes in these two culture systems are notably different.

Oligodendrocytes are characterized by immunolabeling with mature oligodendrocyte marker, the receptor interacting protein (Rip) shown in green, as well as DAPI immunostaining for cell nuclei shown in blue, as demonstrated in FIG. 19B. FIG. 19C is a partial enlarged image of FIG. 19B which shows that the oligodendrocyte morphology in NM:GCM 1:1, 2:1, and 4:1 media appears to be most similar to oligodendrocyte in the brain. FIG. 19D, which is a quantification result of FIG. 19B, shows that the oligodendrocyte population produced by NM:GCM 1:1, 2:1, 4:1, or NM media is range from 3.5 to 6.5%. Our result consistent with the previous study, in which states that in the adult CNS, oligodendrocyte generation from oligodendroglia precursor cell (OPC) is slowed down and white matter (WM) OPC generate about 20% of total differentiated and myelinating oligodendrocytes in the murine corpus callosum vs. 5% in the cortex.

Primary rat cortical GN mix and NE cultures at 9 days in vitro were pretreated with 0.1 μM 14,15-EET, 10 μM 12-(3-adamantan-1-yl-ureido)-dodecanoic acid (AUDA), or vehicle for 30 min, and then treated with or without 14,15-epoxyeicosa-5(Z)-enoic acid (14,15-EEZE) (EZ) or N-methyl-D-aspartate (NMDA). Representative immunofluorescent images of MAP2 and TUNEL staining in GN mix and NE cultures were treated as indicated for 24 h. Total cell nuclei were counterstained with DAPI. Scale bar=50 μm.

FIG. 20 shows immunostaining images of the GN culture, including the control group, and groups subjected to 14,15-EET, sEH inhibitorAUDA, EZ, NMDA, 14,15-EET and NMDA, AUDA and NMDA, and AUDA, EZ, and NMDA treatment. Neuron dendrites were marked with MAP2 and colored in red, cell nuclei were marked with DAPI and colored in blue, while cell death was marked with TUNEL and colored in green.

FIG. 21 shows immunostaining images of the NE culture, including the control group, and groups subjected to 14,15-EET, AUDA,NMDA, 14,15-EET and NMDA, as well as AUDA and NMDA treatment. Neuron dendrites were marked with MAP2 and colored in red, cell nuclei were marked with DAPI and colored in blue, while cell death was marked with TUNEL and colored in green.

FIG. 22A shows quantification of MAP2⁺ neurite density in FIGS. 20 and 21, while FIG. 23 shows quantification of TUNEL⁺ cells in FIGS. 20 and 21. Both MAP2⁺ neurite density and TUNEL⁺ cells were normalized by the total DAPI number in each quantified microscopic field. Note that NMDA-induced MAP2⁺ neurite reduction and cell death are significantly attenuated by both 14,15-EET and AUDA treatments in the GN cultures, but not in NE cultures. Results indicate that 14,15-EET and sEH inhibitor treatments attenuated NMDA-induced neurotoxicity and hyperexcitation in GN mix but not in NE cultures.

FIG. 22B shows that the oligodendrocyte morphology in NM:GCM 1:1 medium for 14 DIV reveals process branching and forms myelin sheath, which is characterized by immunolabeling with Rip, shown in green enwrapping the axon, which is characterized by neuron axonal marker, NF200 shown in red. The cell nuclei are characterized by immunostaining shown in blue. As compared to the control group, the result of the NMDA group in FIG. 22B reveals that the glutamate receptor against NMDA-induced excitotoxicity disrupts oligodendrocyte process extension and arborization. The result shows that the myelination of neuronal axons by oligodendrocytes can be observed using GN mix culture. Moreover, the response of oligodendrocytes to excitotoxic NMDA treatment can further mimic excitotoxicity-induced demyelination.

### Example 8

To validate the in vitro results, the effect of sEH inhibition on neuronal loss in the hippocampus after excitotoxic insult was investigated in vivo. First examined was the effect of CNS administration of AUDA on kainic acid (KA), an epileptic seizure-inducing substance, i.c.v. injection-induced hippocampal damage in male SD adult rats. The extent of neuronal damage in the dorsal hippocampus was examined 24 h after the KA injection by immunostaining with NeuN and MAP2 for neuronal nuclei and dendrites, respectively. The immunostaining images and quantitative analysis showed that, compared with saline, KA injection significantly reduced NeuN⁺ neurons and the MAP2⁺ dendrite area in the dentate gyrus hilus (DGh) region and Cornu Ammonis 3 (CA3), but not in the Cornu Ammonis 1 (CA1); all these neuronal damage effects were ameliorated by AUDA treatment.

Eight-week-old male SD rats were subjected to unilateral i.c.v. injection of saline, KA, or KA with the sEH inhibitor AUDA. Rat brains were harvested 24 h after the injection for immunohistochemistry. FIG. 24 shows representative immunofluorescent images of different hippocampal areas double stained with neuronal nuclei marker NeuN (green) and dendritic marker MAP2 (red). FIG. 25 shows representative immunofluorescent images of different hippocampal areas with GLT-1 staining (green) counterstained with DAPI (blue). Notably, excitotoxicity induced neuronal loss in the stratum pyramidale (SP) and astrocyte hypertrophy in the stratum raditum (SR) in rodent hippocampal CA3.

FIG. 26 shows quantitative analyses of images in FIG. 24. NeuN⁺ cells of each hippocampal subregion were determined by counting the number of NeuN⁺/DAPI⁺ cells and then normalized with respective saline treatment group. MAP2 area was determined by the percentage of the immunoreactive area in each hippocampal region of interest and then normalized with the respective saline treatment group. Note that AUDA treatment significantly attenuated the KA-induced NeuN⁺ cell loss and MAP2⁺ dendrite damage in DGh and CA3. Scale bar=100 μm. When compared with FIGS. 20-23, it can be seen that the results of in vivo trials were more similar to those of the GN culture than the NE culture.

FIG. 27 is a quantitative analysis of images in FIG. 25. GLT-1 area was determined by the percentage of the immunoreactive area in each hippocampal region of interest and then normalized with the respective saline treatment group. Note that GLT-1 immunoreactivity was depleted in the stratum pyramidale (SP) and stratum lucidum (SL) layers of CA3 in the KA treatment group, and the effect was attenuated by the AUDA pretreatment.

### Example 9

To validate the results of in vitro LPS treatment of the present invention, LPS was injected intraperitoneally in mice for 7 days at 3 mg/kg body weight per injection and the astrocyte and microglia plasticity in mouse hippocampi examined. FIG. 28 shows immunofluorescent images of CA1 and CA3 subregions of the hippocampus, injected with saline or LPS. Astrocytes were marked with GFAP and colored in green, while cell nuclei were marked with DAPI and colored in blue. As can be seen, LPS induced astrocyte hypertrophy and proliferation in mouse hippocampal CA1 and CA3, similar to the in vitro results of FIGS. 7, 10, and 11.

FIG. 29 shows immunofluorescent images of the CA1 subregion of the hippocampus, injected with saline or LPS. Microglia were marked with Iba-1 and colored in green, while cell nuclei were marked with DAPI and colored in blue. As can be seen, LPS induced microglial hypertrophy and proliferation in mouse hippocampal CA1, similar to the in vitro results of FIGS. 15, 16, and 17.

Although the present invention has been described in terms of specific exemplary embodiments and examples, it can be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims. It is to be understood that both the foregoing general description and the following detailed description are exemplary and explanatory only and are not restrictive of the invention, as claimed.

These and other changes can be made to the technology in light of the detailed description. In general, the terms used in the following disclosure should not be construed to limit the technology to the specific embodiments disclosed in the specification, unless the above detailed description explicitly defines such terms. Accordingly, the actual scope of the technology encompasses the disclosed embodiments and all equivalent ways of practicing or implementing the technology.

What is claimed is:

1. A glia cell and neuron co-culture system comprising glia cells and neurons co-cultured in a combination of glia culture medium and neuron medium wherein the glia culture medium comprises Dulbecco's modified eagle medium (DMEM) and the neuron medium comprises Neurobasal™ medium.

2. The system of claim 1, wherein the glia cells and neurons are obtained from mammal brains.

3. The system of claim 1, wherein the glia cells and neurons have cell morphology, cell reactions and/or cell interactions that exist in vivo after co-culturing for up to about 21 days, up to about 30 days, up to about 40 days, up to about 45 days.

4. The system of claim 1, wherein the co-culture system comprises a 2D culture.

5. The system of claim 1, wherein the neuron medium further comprises about 1000 to about 2000 mg/L glucose and DMEM comprises about 1750 to about 2750 mg/L glucose with no glutamine

6. The system of claim 5, further comprising 50× B-27 Plus™ supplement and 100× GlutaMAX™ supplement.

7. The system of claim 6, wherein the glia culture medium further comprising one or more antibiotic.

8. The system of claim 7, wherein the glia culture medium further comprising one or more animal serum.

9. The system of claim 5, further comprising 1×B-27 serum free supplement and 2 mM L-glutamine.

10. The system of claim 1, wherein the ratio of glia culture medium to the neuron medium is about 0.5:1 to about 4:1.

11. The system of claim 1, wherein the ratio of glia culture medium to the neuron medium is about 0.5:1.

12. The system of claim 1, wherein the ratio of glia culture medium to the neuron medium is about 1:1.2 to about 4:1.

13. The system of claim 1, wherein the glia cells and neurons are co-cultured together starting at days in vitro (DIV)-0 and the cells mature together for more than 2 days, 5 days, 10 days or 21 days.

14. The system of claim 1, wherein the combination glia culture medium and neuron medium does not comprise factors that promote differentiation, reversion or transformation from one cell type to another cell type including retinoid acid, leukemia inhibitory factor or N2 supplement.

15. The system of claim 1, wherein the glia cells comprise the combination of any glia cells that exists in mammal brains.

16. The system of claim 1, wherein the glia cells comprise the combination of glia cells that usually exists in mammal brains and at about the same percentage ranges as that exist in mammal brains.

17. The system of claim 1, wherein the glia cell comprises astrocytes, microglia, oligodendrocytes, stem cells or a combination thereof wherein the stems cells comprise no more than about 95%, 90%, 80%, 70%, 60%, 50%, 40%, 30%, 20%, 10% or 5% of the cells.

18. A method for preparing the system of claim 1, comprising:
(i) triturating glia cells and neurons obtained from brain of an animal in the glia culture medium;
(ii) stationing for 1 minute at room temperature to obtain the supernatant;
(iii) centrifuging the supernatant at 1,500 rpm to obtain its cell pellet;
(iv) resuspending the pellet in a new glia culture medium and seeding the solution in a plate for 1 hour;
(v) and replacing the medium with the glia culture medium and the neuron medium.

19. The method of claim 18, wherein the glia cells and neurons are obtained from hippocampus, cerebellum, midbrain, spinal cord or dorsal root ganglion of the brain

20. A method for using the system of claim 1 in substitution or support of in vivo models using the system of claim 1 as an animal model.

21. The method of claim 20, wherein the system of claim 1 is used as an animal model in studies of oxygen-glucose deprivation, ischemic stroke, inflammation, cell death, gliosis, ischemic stroke, encephalitis, encephalitis, virus infection, Amyotrophic Lateral Sclerosis or neurodegenerative disease, such as Parkinson disease and Alzheimer's disease, vascular dementia, Alzheimer's Disease, and/or Huntington's Disease.
